(19) United States
(12) Patent Application Publication
(10) Pub. No.: US 2011/0294766 A1
(43) Pub. Date: Dec. 1, 2011

(54) 16 Alpha, 17 Alpha-Acetal Glucocorticosteroidal Derivatives and their Use

(75) Inventors: Frank Burkamp, Lund (SE); Tesfaledet Mussie, Lund (SE); Paul Andersson, Sodra Sandby (SE); Inga-Britt Andersson, Sodra Sandby (SE); Sumita Bhatacharyya, Sodertalje (SE); Håkan Bladh, Lund (SE); Svetlana Ivanova, Lund (SE); Matti Lepistö, Lund (SE)

(73) Assignee: AstraZeneca AB, Sodertalje (SE)

(21) Appl. No.: 12/919,160

(22) PCT Filed: Feb. 27, 2009
(86) PCT No.: PCT/SE2009/050220

§ 371 Date: Jan. 14, 2011

Related U.S. Application Data
(60) Provisional application No. 61/031,801, filed on Feb. 27, 2008.

(51) Int. Cl. A61K 31/58; A61P 37/08; A61P 11/06; C07J 71/00; A61P 11/00

(52) U.S. Cl. 514/171; 540/67; 514/174

(57) ABSTRACT

The present invention provides compounds of formula (I) wherein R¹, R², R³, R⁴, R⁵ and R⁶ are as defined in the specification, a process for their preparation, pharmaceutical compositions containing them and their use in therapy.

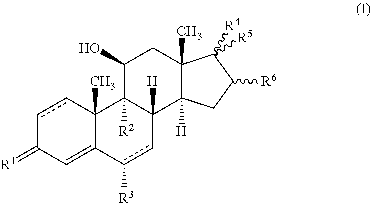

The present invention relates to compounds having glucocorticosteroid receptor agonist activity, processes for their preparation, pharmaceutical compositions containing them and their therapeutic use, particularly for the treatment of inflammatory and allergic conditions.

Glucocorticosteroids (GCs) that have anti-inflammatory properties are known and are widely used for the treatment of diseases such as inflammatory arthritides (e.g. rheumatoid arthritis, ankylosing spondylitis and psoriatic arthropathy), other rheumatoid diseases such as systemic lupus erythematosis, scleroderma, vascutitides including temporal arteritis and polyarteritis nodosa, inflammatory bowel disease such as Crohns disease and ulcerative colitis, lung diseases such as asthma and chronic obstructive airways disease, as well as many other conditions such as polymyalgia rheumatica. GCs have also been used very extensively for their immunosuppressive properties in the prevention and treatment of transplant rejection. Finally GCs have been used for their anti-tumour effects in a number of malignancies.

GCs act via specific glucocorticoid receptors (GR) that are members of the nuclear receptor superfamily. Ligand binding promotes receptor dimerisation, DNA binding, and transcriptional activation. This mechanism of GC action is well defined in vitro and is critical for regulation of the hypothalamic-pituitary-adrenal axis, gluconeogenesis as well as transcription of anti-inflammatory genes such as mitogen-activated protein kinase phosphatase-1 (MKP-1) and secretory leukocyte protease inhibitor (SLPI) in vivo. Ligand-bound receptor is also able to suppress gene transcription in a dimerisation-independent manner by interfering with the activity of transcription factors, such as AP-1 and NFkB, which are critically involved in the inflammatory reaction.

After ligand binding, the GR translocates from the cytoplasm of the cell to the nucleus and binds to glucocorticoid response elements in regulator regions of target genes. The activated GR then recruits co-factors, including the glucocorticoid receptor interacting protein 1 (GRIP-1) and steroid receptor co-activator 1 (SRC1). These accessory proteins bind to the receptor and link the GR with the general transcription machinery to drive transcription of target genes.

Glucocorticoid effects on transcription may be mediated by both the direct binding of activated GR to target DNA, homodimerisation and recruitment of co-activators (known as “transactivation”) but also by GR interfering with other transcription factor function, including AP-1 and NFkB, by complexing with these other transcription factors and preventing them from binding to their target genes leading to repression of the genes normally upregulated by AP-1 or NFkB (known as “transrepression”). These two modes of receptor activity are dissociable and negative effects on NFkB activity can be retained in the absence of transactivation. It appears that transrepression is largely responsible for mediating the therapeutically desirable anti-inflammatory activity of the GR. Interestingly, the IC₅₀ for inhibition of AP-1 or NFkB (0.04 nM) is lower than the EC₅₀ for activation of target genes (5 nM) and yet high doses of GCs are frequently required to treat patients with inflammatory disease. One explanation is that cytokines expressed at the site of inflammation may induce relative glucocorticoid resistance, for instance by activating AP-1 or NFkB. This is of importance as many pro-inflammatory cytokines signal by activation of NFkB and a major anti-inflammatory action of GCs is thought to be mediated by opposing NFkB action.

In accordance with the present invention, there is provided a compound of formula

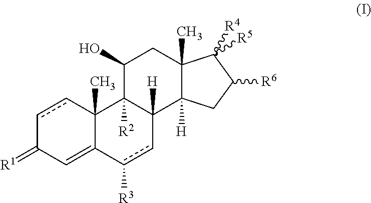

wherein
R¹ represents an oxygen atom;
R² represents a hydrogen, fluorine or chlorine atom;
R³ represents a hydrogen, fluorine or chlorine atom or a methyl group;
R⁴ represents —C(O)—Y—R⁷;
Y represents an oxygen or sulphur atom or a group >NR⁸;
R⁵ and R⁶ together with the carbon atoms to which they are attached form a 1,3-dioxolanyl group which is substituted by a 5- to 10-membered aromatic or heteroaromatic ring system optionally attached to the 1,3-dioxolanyl group via an alkylene, alkenylene or alkynylene linking group, the ring system itself being optionally substituted by one or more substituents independently selected from halogen, cyano, C₁-C₆ alkyl, C₁-C₆ alkoxy, trifluoromethyl and trifluoromethoxy;
R⁷ represents a C₁-C₆ alkyl, C₂-C₆ alkenyl or C₂-C₆ alkynyl group, each of which may be optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₘR⁹, —NHR¹⁰, and —NR¹¹R¹²;
m is 0, 1 or 2;
R⁸ represents a hydrogen atom, a group R⁷, or is linked to R⁷ to form a 3- to 8-membered, saturated or partially saturated heterocyclic ring optionally containing a further ring heteroatom selected from nitrogen, oxygen and sulphur, the heterocyclic ring being optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₙR¹³ and —NR¹⁴R¹⁵;
n is 0, 1 or 2;
R⁹, R¹⁰, R¹¹, R¹², R¹³, R¹⁴ and R¹⁵ each independently represent a C₁-C₆ alkyl group or an aryl group, each of which may be optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₚR¹⁶ and —NR¹⁷R¹⁸, and R¹⁴ and R¹⁵ may each additionally represent a hydrogen atom;
p is 0, 1 or 2; and
R¹⁶, R¹⁷ and R¹⁸ each independently represent a hydrogen atom or a C₁-C₆ alkyl group;
   or a pharmaceutically acceptable salt thereof.

According to one aspect of the invention, the compounds of formula (I) are those in which:
R¹ represents an oxygen atom;
R² represents a hydrogen, fluorine or chlorine atom;
R³ represents a hydrogen, fluorine or chlorine atom or a methyl group;
R⁴ represents —C(O)—Y—R⁷;
Y represents an oxygen or sulphur atom or a group >NR⁸;
R⁵ and R⁶ together with the carbon atoms to which they are attached form a 1,3-dioxolanyl group which is substituted by a 5- to 10-membered heteroaromatic ring system optionally attached to the 1,3-dioxolanyl group via an alkylene, alkenylene or alkynylene linking group;
R⁷ represents a C₁-C₆ alkyl, C₂-C₆ alkenyl or C₂-C₆ alkynyl group, each of which may be optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₘR⁹, —NHR¹⁰, and —NR¹¹R¹²;
m is 0, 1 or 2;
R⁸ represents a hydrogen atom, a group R⁷, or is linked to R⁷ to form a 3- to 8-membered, saturated or partially saturated heterocyclic ring optionally containing a further ring heteroatom selected from nitrogen, oxygen and sulphur, the heterocyclic ring being optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₙR¹³ and —NR¹⁴R¹⁵;
n is 0, 1 or 2;
R⁹, R¹⁰, R¹¹, R¹², R¹³, R¹⁴ and R¹⁵ each independently represent a C₁-C₆ alkyl group or an aryl group, each of which may be optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₚR¹⁶ and —NR¹⁷R¹⁸, and R¹⁴ and R¹⁵ may each additionally represent a hydrogen atom;
p is 0, 1 or 2; and
R¹⁶, R¹⁷ and R¹⁸ each independently represent a hydrogen atom or a C₁-C₆ alkyl group.

In the context of the present specification, unless otherwise stated, an alkyl, alkenyl or alkynyl substituent group or an alkyl, alkenyl or alkynyl moiety in a substituent group may be linear or branched. Examples of C₁-C₆ alkyl groups/moieties include methyl, ethyl, propyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, tert-butyl, n-pentyl, isopentyl, neopentyl and n-hexyl. Examples of C₂-C₆ alkenyl and C₂-C₆ alkynyl groups/moieties include ethenyl, propenyl, 1-butenyl, 2-butenyl, 1-pentenyl, 1-hexenyl, 1,3-butadienyl, 1,3-pentadienyl, 1,4-pentadienyl, 1-hexadienyl, ethynyl, propynyl, 1-butynyl, 2-butynyl, 1-pentynyl and 1-hexynyl. Similarly, an alkylene, alkenylene or alkynylene linking group may be cyclic, linear or branched and may contain, for example, up to a total of eight carbon atoms. Examples of C₁-C₆ alkylene linking groups include methylene, ethylene, n-propylene, n-butylene, n-pentylene, n-hexylene, 1-methylethylene, 2-methylethylene, 1,2-dimethylethylene, 1-ethylethylene, 2-ethylethylene, 1-, 2- or 3-methylpropylene and 1-, 2- or 3-ethylpropylene; C₂-C₆ alkenylene linking groups containing one or more carbon-carbon double bonds include vinylidene, ethenylene (vinylene), propenylene, methylethenylene, 1-propenylidene, 2-propenylidene, 3-methylpropenylene, 3-ethylpropenylene, 1,3-dimethylpropenylene, 2,3-dimethylpropenylene, 3,3-dimethylpropenylene, 3-ethyl-1-methylpropenylene, 1,3,3-trimethylpropenylene and 2,3,3-trimethylpropenylene; and C₂-C₆ alkynylene linking groups containing one or more carbon-carbon triple bonds include ethynylene, propynylene, and 2-butynylene. A C₁-C₆ haloalkyl or C₁-C₆ haloalkoxy substituent group/moiety will comprise at least one halogen atom, e.g. one, two, three, four or five halogen atoms, examples of which include trifluoromethyl, trifluoromethoxy or pentafluoroethyl. A C₁-C₆ hydroxyalkyl substituent group/moiety will comprise at least one hydroxyl group, e.g. one, two, three or four hydroxyl groups, examples of which include —CH₂OH, —CH₂CH₂OH, —CH₂CH₂CH₂OH and —CH(CH₂OH)₂. For the avoidance of doubt, it should be understood that in R⁸, the definition of the “heterocyclic ring” is not intended to include unstable structures or any 0-0 or O—S bonds and that a substituent, if present, may be attached to any suitable ring atom.

An “aryl” group refers to a mono-, bi- or tri-cyclic carbocyclic aromatic radical, and includes radicals having two monocyclic carbocyclic aromatic rings that are directly linked by a covalent bond. Illustrative of such radicals are phenyl, naphthyl, biphenyl, fluorenyl and indenyl.

When any chemical moiety or group in formula (I) is described as being optionally substituted, it will be appreciated that the moiety or group may be either unsubstituted or substituted by one or more of the specified substituents. It will be appreciated that the number and nature of substituents will be selected so as to avoid sterically undesirable combinations.

In an embodiment of the invention, R² represents a hydrogen or a fluorine atom, particularly a fluorine atom.

In an embodiment of the invention, R³ represents a hydrogen or a fluorine atom, particularly a fluorine atom.

When R³ represents a methyl group, the ring carbon atom to which R³ is attached may be unsaturated as illustrated in the following structural formula:

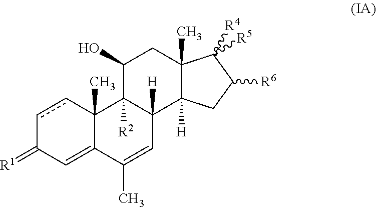

where R¹, R², R⁴, R⁵ and R⁶ are as defined in formula (I).

R⁴ represents —C(O)—Y—R⁷ where Y represents an oxygen or sulphur atom or a group >NR⁸, preferably a sulphur atom, and R⁷ is as defined above.

R⁵ and R⁶ together with the carbon atoms to which they are attached form a 1,3-dioxolanyl group which is substituted by a 5- to 10-, or 5- to 9-, or 5- to 6-, membered aromatic or heteroaromatic ring system optionally attached to the 1,3-dioxolanyl group via an alkylene, alkenylene or alkynylene linking group, the linking group preferably containing up to a total of 8 carbon atoms, e.g. from 1 to 6 or 1 to 4 carbon atoms, the ring system itself being optionally substituted by one or more (e.g. on, two, three or four, particularly one or two) substituents independently selected from halogen (e.g. fluorine, chlorine, bromine or iodine), cyano, C₁-C₆, or C₁-C₄, or C₁-C₂ alkyl, C₁-C₆, or C₁-C₄, or C₁-C₂ alkoxy, trifluoromethyl and trifluoromethoxy. Preferred substituents on the aromatic or heteroaromatic ring system include fluorine, chlorine, cyano, methyl, methoxy, trifluoromethyl and trifluoromethoxy.

The aromatic or heteroaromatic ring system may be a monocyclic, bicyclic (e.g. a 6,6- or 6,5-fused bicyclic) or tricyclic ring system and includes radicals having two such monocyclic rings or one such monocyclic ring and one monocyclic aryl ring which are directly linked by a covalent bond. The heteroaromatic ring system will contain one or more ring heteroatoms independently selected from nitrogen, oxygen and sulphur. Examples of such aromatic and heteroaromatic ring systems include phenyl, naphthyl, biphenyl, fluorenyl, indenyl, pyridinyl, pyrimidinyl, triazolyl, benztriazolyl, thiadiazolyl, oxadiazolyl, pyridazinyl, pyrazinyl, triazinyl, thiazolyl, benzthiazolyl, oxazolyl, isoxazolyl, thienyl, pyrazolyl, imidazolyl, benzimidazolyl, furanyl, 2,3-dihydrobenzofuranyl, benzofuranyl, isoxazolyl, benzisoxazolyl, pyrrolyl, isothiazolyl, benzisothiazolyl, quinolinyl, isoquinolinyl, indolyl, benzothiophenyl, 1H-indazolyl, benzoxazolyl, purinyl, cinnolinyl, phthalazinyl, quinazolinyl, quinoxalinyl, 1,8-naphthyridinyl and pteridinyl. Preferred aromatic or heteroaromatic ring systems include phenyl, furanyl, thienyl, benzofuranyl, quinolinyl, 2,3-dihydrobenzofuranyl and isoxazolyl, particularly phenyl, furanyl and thienyl.

In an embodiment of the invention, R⁵ and R⁶ together with the carbon atoms to which they are attached form a 1,3-dioxolanyl group which is substituted by a 5- to 6-membered aromatic or heteroaromatic ring system optionally attached to the 1,3-dioxolanyl group via an alkylene, alkenylene or alkynylene linking group, the ring system itself being optionally substituted by one or two substituents independently selected from fluorine, chlorine, cyano, methyl, methoxy, trifluoromethyl and trifluoromethoxy.

In an embodiment of the invention, R⁵ and R⁶ together with the carbon atoms to which they are attached form a 1,3-dioxolanyl group which is substituted by a 5-membered heteroaromatic ring, the heteroaromatic ring itself being optionally substituted by one or two substituents independently selected from chlorine, methyl or trifluoromethyl.

In a further embodiment, R⁵ and R⁶ together with the carbon atoms to which they are attached form a 1,3-dioxolanyl group which is substituted by a furanyl, thienyl or isoxazolyl group, each of which may be optionally substituted by one or two substituents independently selected from chlorine, methyl or trifluoromethyl.

In a still further embodiment, R⁵ and R⁶ together with the carbon atoms to which they are attached form a 1,3-dioxolanyl group which is substituted by a phenyl group, the phenyl group itself being optionally substituted by one or two substituents independently selected from fluorine, chlorine, cyano, methyl, methoxy, trifluoromethyl and trifluoromethoxy.

R⁷ represents a C₁-C₆, or C₁-C₄, alkyl, C₂-C₆, or C₂-C₄, alkenyl or C₂-C₆, or C₂-C₄, alkynyl group, each of which may be optionally substituted by one or more (e.g. one, two, three or four) substituents independently selected from hydroxyl, halogen (e.g. fluorine, chlorine, bromine or iodine), cyano, nitro, C₁-C₆, or C₁-C₄, or C₁-C₂ alkyl, C₂-C₆ or C₂-C₄ alkenyl, C₁-C₆, or C₁-C₄, or C₁-C₂ haloalkyl, C₁-C₆, or C₁-C₄, or C₁-C₂ hydroxyalkyl, C₁-C₆, or C₁-C₄, or C₁-C₂ alkoxy, C₁-C₆, or C₁-C₄, or C₁-C₂ haloalkoxy, C₁-C₆, or C₁-C₄, or C₁-C₂ alkylcarbonyl, C₁-C₆, or C₁-C₄, or C₁-C₂ alkylcarbonyloxy, C₁-C₆, or C₁-C₄, or C₁-C₂ alkoxycarbonyl, —S(O)ₘR⁹, —NHR¹⁰, and —NR¹¹R¹².

In one embodiment, R⁷ represents a C₁-C₃ alkyl (particularly methyl), C₂-C₄ alkenyl or C₂-C₄ alkynyl (particularly a butynyl such as 2-butynyl) group, each of which may be optionally substituted by one or more (e.g. one, two, three or four) substituents independently selected from hydroxyl, fluorine, chlorine, cyano, nitro, C₁-C₄ alkyl, C₂-C₄ alkenyl, C₁-C₄ haloalkyl, C₁-C₄ hydroxyalkyl, C₁-C₄ alkoxy, C₁-C₄ haloalkoxy, C₁-C₄ alkylcarbonyl, C₁-C₄ alkylcarbonyloxy, C₁-C₄ alkoxycarbonyl, —S(O)ₙR⁹, —NHR¹⁰, and —NR¹¹R¹².

In yet another embodiment, R⁷ represents a methyl or a butynyl group, each of which may be optionally substituted by a hydroxyl, fluorine or cyano group.

R⁸ represents a hydrogen atom, a group R⁷, or is linked to R⁷ to form a 3- to 8-membered, or 3- to 6-membered, saturated or partially saturated nitrogen-containing heterocyclic ring optionally containing a further ring heteroatom (eg. one, two or three ring heteroatoms independently) selected from nitrogen, oxygen and sulphur, the heterocyclic ring being optionally substituted by one or more (e.g. one, two, three or four) substituents independently selected from hydroxyl, halogen (e.g. fluorine, chlorine, bromine or iodine), cyano, nitro, C₁-C₆, or C₁-C₄, or C₁-C₂ alkyl, C₂-C₆ or C₂-C₄ alkenyl, C₁-C₆, or C₁-C₄, or C₁-C₂ haloalkyl, C₁-C₆, or C₁-C₄, or C₁-C₂ hydroxyalkyl, C₁-C₆, or C₁-C₄, or C₁-C₂ alkoxy, C₁-C₆, or C₁-C₄, or C₁-C₂ haloalkoxy, C₁-C₆, or C₁-C₄, or C₁-C₂ alkylcarbonyl, C₁-C₆, or C₁-C₄, or C₁-C₂ alkylcarbonyloxy, C₁-C₆, or C₁-C₄, or C₁-C₂ alkoxycarbonyl, —S(O)ₙR¹³ and —NR¹⁴R¹⁵.

Examples of 3- to 8-membered saturated or partially saturated heterocyclic rings include morpholine, azetidine, pyrrolidine, piperidine, piperazine, 3-pyrroline and thiomorpholine.

R⁹, R¹⁰, R¹¹, R¹², R¹³, R¹⁴ and R¹⁵ each independently represent a C₁-C₆, or C₁-C₄, alkyl group or an aryl group, each of which may be optionally substituted by one or more (e.g. one, two, three or four) substituents independently selected from hydroxyl, halogen (e.g. fluorine, chlorine, bromine or iodine), cyano, nitro, C₁-C₆, or C₁-C₄, or C₁-C₂ alkyl, C₂-C₆ or C₂-C₄ alkenyl, C₁-C₆, or C₁-C₄, or C₁-C₂ haloalkyl, C₁-C₆, or C₁-C₄, or C₁-C₂ hydroxyalkyl, C₁-C₆, or C₁-C₄, or C₁-C₂ alkoxy, C₁-C₆, or C₁-C₄, or C₁-C₂ haloalkoxy, C₁-C₆, or C₁-C₄, or C₁-C₂ alkylcarbonyl, C₁-C₆, or C₁-C₄, or C₁-C₂ alkylcarbonyloxy, C₁-C₆, or C₁-C₄, or C₁-C₂ alkoxycarbonyl, —S(O)ₚR¹⁶ and —NR¹⁷R¹⁸, and R¹⁴ and R¹⁵ may each additionally represent a hydrogen atom.

R¹⁶, R¹⁷ and R¹⁸ each independently represent a hydrogen atom or a C₁-C₆, or C₁-C₄, or C₁-C₂ alkyl group.

In an embodiment of the invention, the compounds have the following structural formula:

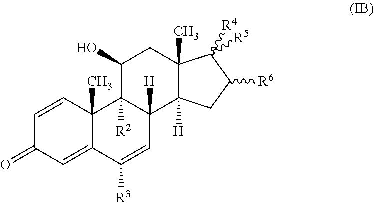

wherein R², R³, R⁴, R⁵ and R⁶ are as defined above.

In one aspect, the invention provides a compound of formula (I), (IA) or (IB), or a pharmaceutically acceptable salt thereof, in which:
R¹ represents an oxygen atom;
R² represents a hydrogen or fluorine atom;
R³ represents a hydrogen or fluorine atom;
R⁴ represents —C(O)—Y—R⁷;
Y represents a sulphur atom;
R⁵ and R⁶ together with the carbon atoms to which they are attached form a 1,3-dioxolanyl group which is substituted by a 5- to 10-membered aromatic or heteroaromatic ring system, the ring system itself being optionally substituted by one or more substituents independently selected from halogen, cyano, C₁-C₆ alkyl, C₁-C₆ alkoxy, trifluoromethyl and trifluoromethoxy; and
R⁷ represents a C₁-C₆ alkyl or C₂-C₆ alkynyl group, each of which may be optionally substituted by one or more substituents independently selected from hydroxyl, halogen or cyano.

Examples of compounds of the invention include:
S-(Cyanomethyl)(4aS,4bR,6aS,6bS,9aR,10aS,10bS,12S)-4b,12-difluoro-8-(2-furyl)-4a,6a-dimethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carbothioate,
S-(4-Hydroxybut-2-yn-1-yl)(4aS,4bR,6aS,6bS,9aR,10aS,10bS,12S)-4b,12-difluoro-8-(2-furyl)-4a,6a-dimethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carbothioate,
S-(Fluoromethyl)(4aS,4bR,6aS,6bS,9aR,10aS,10bS,12S)-4b,12-difluoro-8-(2-furyl)-4a,6a-dimethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carbothioate,
S-Methyl (4aS,4bR,5S,6aS,6bS,8S,9aR,10aS,10bS,125)-4b,12-difluoro-5-hydroxy-4a,6a-dimethyl-2-oxo-8-(2-thienyl)-2,4a,4b,5,6,6a,9a,10,10a,10b,11,12-dodecahydro-6bH-naphtho[2′,1′:4,5]indeno[1,2-d][1,3]dioxole-6b-carbothioate,
S-Methyl (4aS,4bR,5S,6aS,6bS,9aR,10aS,10bS,12S)-8-(2,4-difluorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,125)-8-(2,4-difluorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,8S,9aR,10aS,10bS,125)-4b,12-difluoro-5-hydroxy-4a,6a-dimethyl-8-(5-methyl-2-furyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-Methyl (4aS,4bR,5S,6aS,6bS,9aR,10aS,10bS,125)-4b,12-difluoro-5-hydroxy-4a,6a-dimethyl-8-(5-methyl-2-furyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,125)-4b,12-difluoro-5-hydroxy-4a,6a-dimethyl-2-oxo-8-(2-thienyl)-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-5-hydroxy-4a,6a-dimethyl-8-(5-methyl-2-furyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-8-(5-chloro-2-furyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-8-(1-benzofuran-2-yl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-8-(3-methyl-2-thienyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-2-oxo-8-[5-(trifluoromethyl)-2-furyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-2-oxo-8-(2-thienyl)-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-5-hydroxy-4a,6a-dimethyl-2-oxo-8-quinolin-6-yl-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-8-(4-methylphenyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,125)-4b,12-difluoro-5-hydroxy-4a,6a-dimethyl-2-oxo-8-[4-(trifluoromethoxy)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-8-(2-methylphenyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-8-(3-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-5-hydroxy-4a,6a-dimethyl-8-(4-methylphenyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-8-(3-cyanophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-8-(4-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-8-(4-chlorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-8-(4-chlorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-2-oxo-8-phenyl-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-5-hydroxy-4a,6a-dimethyl-2-oxo-8-phenyl-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-8-(2,3-dihydro-1-benzofuran-7-yl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-8-(3,5-dimethylisoxazol-4-yl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aR,4bS,5S,6aS,6bS,9aR,10aS,10bS)-8-furan-2-yl-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aR,4bS,5S,6aS,6bS,8R,9aR,10aS,10bS)-5-hydroxy-4a,6a-dimethyl-2-oxo-8-thiophen-2-yl-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aR,4bS,5S,6aS,6bS,9aR,10aS,10bS)-8-(2,4-dimethylphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aR,4bS,5S,6aS,6bS,9aR,10aS,10bS)-8-(2-fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-Methyl (4aR,4bS,5S,6aS,6bS,8R,9aR,10aS,10bS)-8-(2-fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-Methyl (4aR,4bS,5S,6aS,6bS,8S,9aR,10aS,10bS)-8-(2-fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aR,4bS,5S,6aS,6bS,9aR,10aS,10bS)-5-hydroxy-4a,6a-dimethyl-2-oxo-8-[4-(trifluoromethyl)phenyl]-2,4a,4b,5,6,6a,9a,10,10a, 10b,11,12-dodecahydro-6bH-naphtho[2′,1′:4,5]indeno[1,2-d][1,3]dioxole-6b-carbothioate,
S-(Fluoromethyl)(4aR,4bS,5S,6aS,6bS,9aR,10aS,10bS)-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-Methyl (4aR,4bS,5S,6aS,6bS,9aR,10aS,10bS)-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aR,4bS,5S,6aS,6bS,9aR,10aS,10bS)-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aR,4bS,5S,6aS,6bS,9aR,10aS,10bS)-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate, and
S-Methyl (4aR,4bS,5S,6aS,6bS,9aR,10aS,10bS)-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate.

It should be noted that each of the chemical compounds listed above represents a particular and independent aspect of the invention.

The present invention further provides a process for the preparation of a compound of formula (I) or a pharmaceutically acceptable salt thereof as defined above which comprises

(i) reacting a compound of formula (II)

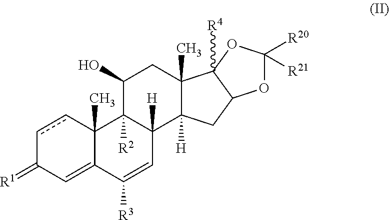

where R²⁰ and R²¹ each independently represent a C₁-C₆ alkyl (e.g. methyl) group and R¹, R², R³ and R⁴ are as defined in formula (I), with a compound of formula (III), OCH—X—R²², where X represents a bond or an alkylene, alkenylene or alkynylene linking group and R²² represents a 5- to 10-membered aromatic or heteroaromatic ring system optionally substituted by one or more substituents independently selected from halogen, cyano, C₁-C₆ alkyl, C₁-C₆ alkoxy, trifluoromethyl and trifluoromethoxy, or
(ii) when Y represents a sulphur atom, hydrolysing a compound of formula (IV)

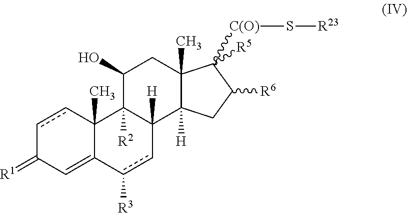

where R²³ represents a sulphur-protecting group (e.g. —C(O)N(CH₃)₂) and R¹, R², R³, R⁵ and R⁶ are as defined in formula (I), followed by reaction with a compound of formula (V), R⁷-L, where L represents a leaving group (e.g. a halogen atom) and R⁷ is as defined in formula (I), and optionally thereafter carrying out one or more of the following procedures:
converting a compound of formula (I) into another compound of formula (I)
removing any protecting groups
forming a pharmaceutically acceptable salt.

The process (i) above is conveniently carried out in the presence of an organic solvent such as dichloromethane, acetonitrile or dichloroethane at a temperature in the range from, for example, 25° C. to 35° C. Furthermore, it may be desirable to add a catalyst to the reaction such as 1-butyl-3-methylimidazolium hexafluorophosphate (BMIMPF₆) and perchloric acid.

Compounds of formula (II) are either commercially available, are well known in the literature or may be prepared easily using known techniques, see, for example, WO 2004/052912 and WO 2007/054974.

The process (ii) above is conveniently carried out in the presence of an organic solvent at a temperature in the range from, for example, 25° C. to 35° C. The hydrolysis reaction is conveniently carried out in the presence of an organic solvent such as methanol or N,N-dimethylacetamide using an appropriate hydrolysing agent, e.g. potassium carbonate or sodium hydrogensulfide. Subsequent reaction with the compound of formula (V) is conveniently carried out in the presence of an organic solvent such as acetonitrile.

Compounds of formulae (III), (IV) and (V) are either commercially available, are well known in the literature or may be prepared easily using known techniques.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt thereof, preferably an acid addition salt such as a hydrochloride, hydrobromide, trifluoroacetate, sulfate, phosphate, acetate, fumarate, maleate, tartrate, lactate, citrate, pyruvate, succinate, oxalate, methanesulphonate or p-toluenesulphonate.

The compounds of formula (I) and pharmaceutically acceptable salts thereof may exist in solvated, for example hydrated, as well as unsolvated forms, and the present invention encompasses all such solvated forms.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically and diastereomerically pure forms are particularly desired.

The compounds of formula (I) and their pharmaceutically acceptable salts have activity as pharmaceuticals, in particular as modulators of glucocorticoid receptor activity, and thus may be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
3. eyes: blepharitis; conjunctivitis, including perennial and vernal allergic conjunctivitis; iritis; anterior and posterior uveitis; choroiditis; autoimmune, degenerative or inflammatory disorders affecting the retina; ophthalmitis including sympathetic ophthalmitis; sarcoidosis; infections including viral, fungal, and bacterial;
4. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
5. allograft rejection: acute and chronic following, for example, transplantation of kidney, heart, liver, lung, bone marrow, skin or cornea or following blood transfusion; or chronic graft versus host disease;
6. other auto-immune and allergic disorders including rheumatoid arthritis, irritable bowel syndrome, systemic lupus erythematosus, multiple sclerosis, Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome and Sazary syndrome;
7. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
8. infectious diseases: virus diseases such as genital warts, common warts, plantar warts, hepatitis B, hepatitis C, herpes simplex virus, molluscum contagiosum, variola, human immunodeficiency virus (HIV), human papilloma virus (HPV), cytomegalovirus (CMV), varicella zoster virus (VZV), rhinovirus, adenovirus, coronavirus, influenza, para-influenza; bacterial diseases such as tuberculosis and mycobacterium avium, leprosy; other infectious diseases, such as fungal diseases, chlamydia, candida, aspergillus, cryptococcal meningitis, pneumocystis carnii, cryptosporidiosis, histoplasmosis, toxoplasmosis, trypanosome infection and leishmaniasis.

Thus, the present invention provides a compound of formula (I) or a pharmaceutically acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

In particular, the compounds of the invention (including pharmaceutically acceptable salts) may be used in the treatment of asthma {such as bronchial, allergic, intrinsic, extrinsic or dust asthma, particularly chronic or inveterate asthma (for example late asthma or airways hyper-responsiveness)}, chronic obstructive pulmonary disease (COPD) or allergic rhinitis.

The invention also provides a method of treating, or reducing the risk of, an obstructive airways disease or condition (e.g. asthma or COPD) which comprises administering to a patient in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

Dry powder formulations and pressurized HFA aerosols of the compounds of the invention (that is, compounds of formula (I) and pharmaceutically acceptable salts thereof) may be administered by oral or nasal inhalation. For inhalation, the compound is desirably finely divided. The finely divided compound preferably has a mass median diameter of less than 10 micrometres (μm), and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention (that is, compounds of formula (I) and pharmaceutically acceptable salts thereof) may also be administered in conjunction with other compounds used for the treatment of the above conditions.

The invention therefore further relates to combination therapies wherein a compound of the invention or a pharmaceutical composition or formulation comprising a compound of the invention is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with the following agents: non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention and a receptor antagonist for leukotrienes (LT) B4, LTC4, LTD4, and LTE4 selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention and a proton pump inhibitor (such as omeprazole) or a gastroprotective histamine type 2 receptor antagonist.

The present invention further relates to the combination of a compound of the invention and an antagonist of the histamine type 4 receptor.

The present invention still further relates to the combination of a compound of the invention and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention and an anticholinergic agents including muscarinic receptor (M1, M2, and M3) antagonist such as atropine, hyoscine, glycopyrrolate, ipratropium bromide, tiotropium bromide, oxitropium bromide, pirenzepine or telenzepine.

The present invention still further relates to the combination of a compound of the invention and a beta-adrenoreceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention and a chromone, such as sodium chromoglycate or nedocromil sodium.

The present invention further relates to the combination of a compound of the invention with an agent that modulates a nuclear hormone receptor such as PPARs.

The present invention still further relates to the combination of a compound of the invention together with an immunoglobulin (Ig) or Ig preparation or an antagonist or antibody modulating Ig function such as anti-IgE (for example omalizumab).

The present invention further relates to the combination of a compound of the invention and another systemic or topically-applied anti-inflammatory agent, such as thalidomide or a derivative thereof, a retinoid, dithranol or calcipotriol.

The present invention still further relates to the combination of a compound of the invention and combinations of aminosalicylates and sulfapyridine such as sulfasalazine, mesalazine, balsalazide, and olsalazine; and immunomodulatory agents such as the thiopurines.

The present invention further relates to the combination of a compound of the invention together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention and an agent for the treatment of acute or chronic pain, such as a centrally or peripherally-acting analgesic (for example an opioid or derivative thereof), carbamazepine, phenyloin, sodium valproate, amitryptiline or other anti-depressant agent-s, paracetamol, or a non-steroidal anti-inflammatory agent.

The present invention further relates to the combination of a compound of the invention together with a parenterally or topically-applied (including inhaled) local anaesthetic agent such as lignocaine or a derivative thereof.

A compound of the present invention can also be used in combination with an anti-osteoporosis agent including a hormonal agent such as raloxifene, or a biphosphonate such as alendronate.

The present invention still further relates to the combination of a compound of the invention together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2×7; (xxvii) inhibitor of transcription factor activation such as NFkB, API, or STATS; or (xxviii) a glucocorticoid receptor agonist.

In a further aspect the present invention provides a combination (for example for the treatment of COPD, asthma or allergic rhinitis) of a compound of formula (I) or a pharmaceutically acceptable salt thereof as hereinbefore defined and one or more agents independently selected from:
a selective β₂ adrenoceptor agonist (such as metaproterenol, isoproterenol, isoprenaline, albuterol, salbutamol, formoterol, salmeterol, terbutaline, orciprenaline, bitolterol mesylate, pirbuterol or indacaterol);
a phosphodiesterase inhibitor (such as a PDE4 inhibitor);
a protease inhibitor (such as a neutrophil elastase or matrix metalloprotease MMP-12 inhibitor);
an anticholinergic agent;
a modulator of chemokine receptor function (such as a CCR1 receptor antagonist); and
an inhibitor of kinase function (such as the kinases p38 or IKK).

The invention also provides a pharmaceutical product comprising, in combination, a preparation of a first active ingredient which is a compound of formula (I) or a pharmaceutically acceptable salt thereof as hereinbefore defined, and a preparation of a second active ingredient which is
a selective β₂ adrenoceptor agonist;
a phosphodiesterase inhibitor;
a protease inhibitor;
an anticholinergic agent;
a modulator of chemokine receptor function; or
an inhibitor of kinase function;
   for simultaneous, sequential or separate use in therapy.

In another aspect, the invention provides a kit comprising a preparation of a first active ingredient which is a compound of formula (I) or a pharmaceutically acceptable salt thereof as hereinbefore defined, and a preparation of a second active ingredient which is
a selective β₂ adrenoceptor agonist;
a phosphodiesterase inhibitor;
a protease inhibitor;
an anticholinergic agent;
a modulator of chemokine receptor function; or
an inhibitor of kinase function;
   and instructions for the simultaneous, sequential or separate administration of the preparations to a patient in need thereof.

A compound of the invention can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The present invention will now be further explained by reference to the following illustrative examples in which the following abbreviations are used:
EtOAc ethyl acetate
DMF N,N-dimethylformamide
NaOH sodium hydroxide
Na₂SO₄ sodium sulfate
DMSO dimethylsulfoxide
TFA trifluoroacetic acid
HCl hydrochloric acid
Et₃N triethylamine
NaSH sodium sulfide
NaHCO₃ sodium hydrogen carbonate
MeCN/
CH₃CN acetonitrile
DCM dichloromethane
DMA N,N-dimethylacetamide
THF tetrahydrofuran
aq. aqueous
conc. concentrated
RT room temperature
hrs hours
min. minutes
M molar
LC liquid chromatography
MS mass spectrometry
APCI atmospheric chemical ionisation method
NMR nuclear magnetic resonance
HPLC High performance liquid chromatography

### GENERAL METHODS

NMR spectra were recorded on a Varian Mercury-VX 300 MHz instrument or a Varian Inova 400 MHz instrument. The central peaks of chloroform-d (H 7.26 ppm), acetone-d₆ (H 2.05 ppm), acetonitrile-d₃ (δ_{H} 1.94 ppm) or DMSO-d₆ (H 2.50 ppm) were used as internal references.

The following method was used for LC/MS analysis:

Instrument Agilent 1100; Column Waters Symmetry 2.1×30 mm; Mass APCI; Flow rate 0.7 mL/min; Wavelength 254 nm; Solvent A: water+0.1% TFA; Solvent B: acetonitrile+0.1% TFA; Gradient 15-95%/B 2.7 min, 95% B 0.3 min.

Column chromatography was carried out using silica gel (0.040-0.063 mm, Merck). For preparative HPLC either a Kromasil KR-100-5-C18 column (250×20 mm, Akzo Nobel) and mixtures of acetonitrile/water (0.1% TFA) at a flow rate of 10 ml/min or a XTerra® Prep MS C₁₈ OBD™ Column, 5 μm, 19×50 mm (acetonitrile/water/0.1% NH₃) at a flow rate of 20 ml/min was used. UV=254 nm or 220 nm was used for detection.

Unless stated otherwise, starting materials were commercially available. All solvents and commercial reagents were of laboratory grade and were used as received.

### Intermediate 1

### (4aS,4bR,6aS,6bS,9aR,10aS,10bS,12S)-4b,12-Difluoro-4a,6a,8,8-tetramethyl-2-oxo-2,4a,4b,5,6,6a,9a,10,10a,10b,11,12-dodecahydro-6bH-naphtho[2′1′:4,5]-indeno[1,2-d][1,3]dioxole-6b-carboxylic acid

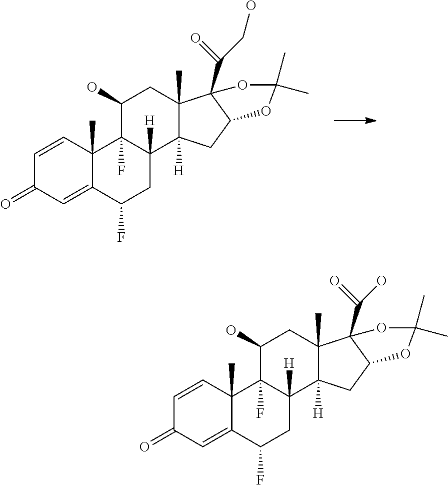

Periodic acid (1.486 g, 6.52 mmol) was added to a solution of Fluocinolone acetonide (2.95 g, 6.52 mmol) in dioxane (20 ml) and water (6 ml). The reaction mixture was stirred at RT in an open flask for 4.5 h, carefully poured into cold saturated aqueous sodium bicarbonate and the mixture was concentrated in vacuo. The residue was partitioned between 100 ml methylene chloride and 100 ml 1M NaOH. The organic phase was discarded and the aqueous phase acidified with concenrated HCl and extracted with 2 times 250 ml EtOAc. After drying over sodium sulfate and filtration, the solvent was evaporated and the residue was dissolved in a minimum amount of EtOAc and precipitated with petroleum ether (40°-60° C.) to give 2.62 g (5.98 mmol, 92%) of the desired product. APCI-MS: m/z 439.2 [MH⁺].

### Intermediate 1 Alternative Method

### (4aS,4bR,6aS,6bS,9aR,10aS,10bS,12S)-4b,12-Difluoro-4a,6a,8,8-tetramethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carboxylic acid

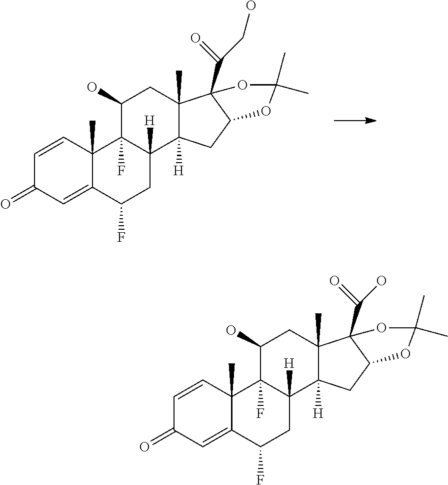

A solution of periodic acid (54.7 g, 240 mmol) in water (80 mL) was added to a solution of Fluocinolone acetonide (54.3 g, 120 mmol) in THF (350 ml) in a 1 L round-bottomed flask at 35° C. The reaction mixture was stirred at 35° C. for 6 days and 80% of the product formation was observed according to LC-MS. The reaction mixture was carefully poured into cold saturated aqueous sodium bicarbonate (1 L) and the mixture was stirred for 1 h. The mixture was washed with ethyl acetate (1.5 L) and 500 ml 1M NaOH was added to the water phase and the mixture was again washed with 1.5 L ethyl acetate. The organic phase was discarded and the clear and colourless alkaline water phase cooled and was then acidified with 5M HCl. The obtained yellow precipitate was filtered, washed 2 times with 500 mL of water and dried to give 36.5 g (69%) of the desired product. APCI-MS: m/z 439.2 [MH⁺].

### Intermediate 2

### S-{[4aS,4bR,6aS,6bS,9aR,10aS,10bS,12S)-4b,12-Difluoro-4a,6a,8,8-tetramethyl-2-oxo-2,4a,4b,5,6,6a,9a,10,10a,10b,11,12-dodecahydro-6bH-naphtho[2′,1′:4,5]-indeno[1,2-d][1,3]dioxol-6b-yl]carbonyl}dimethylthiocarbamate

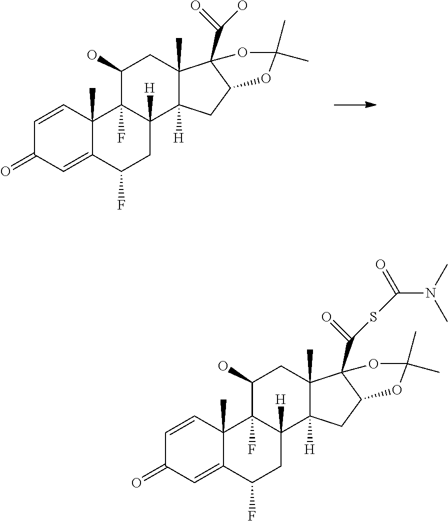

A solution of Intermediate 1 (2.62 g, 5.98 mmol) and N,N-dimethylthiocarbamoyl chloride (2.22 g, 17.93 mmol) in acetone (20 ml) was treated with triethylamine (2.5 ml, 17.93 mmol), sodium iodide (0.22 g, 1.48 mmol) and water (10 ml). The mixture was stirred for 4 hrs at RT. After completion of the reaction N,N-dimethyl acetamide (10 ml) was added to the reaction mixture and stirred further for 30 min. The precipitate obtained was filtered, washed with water and dried in vacuo at 70° C. to give 2.70 g (86%) of the desired compound. APCI-MS: m/z 526 [MH⁺].

### Intermediate 3

### (4aS,4bR,6aS,6bS,9aR,10aS,10bS,12S)-4b,12-Difluoro-4a,6a,8,8-tetramethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carbothioic S-acid

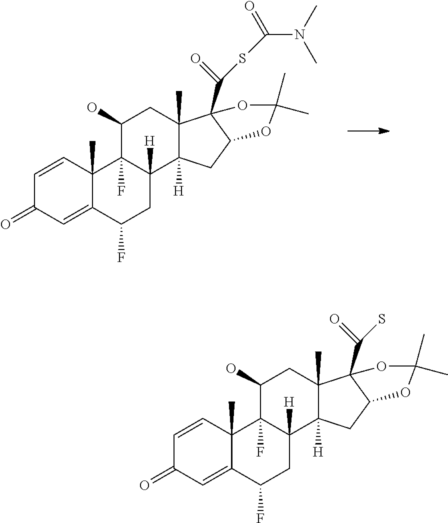

A suspension of Intermediate 2 (2.62 g, 4.98 mmol) and potassium carbonate (1.4 g, 9.97 mmol) in methanol (15 ml) was stirred at RT for 3 hrs. Water was added and the solution was washed with toluene. The aqueous phase was acidified with 2N HCl to ˜1.0 pH and the resulting precipitate was filtered, washed with water and dried in air to give 1.81 g (57%) of the title compound. APCI-MS: m/z 455.2 [MH⁺].

### Intermediate 4

### S-(Cyanomethyl)(4aS,4bR,6aS,6bS,9aR,10aS,10bS,12S)-4b,12-Difluoro-4a,6a,8,8-tetramethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carbothioate

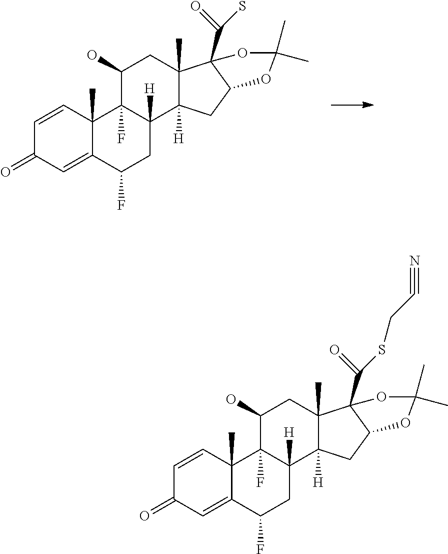

To a solution of Intermediate 3 (870 mg, 1.91 mmol) in acetone (10 ml) was added potassium carbonate (529 mg, 3.83 mmol) and bromoacetonitrile (0.153 ml, 2.30 mmol) and the mixture was stirred for 4 hrs. The reaction mixture was poured into EtOAc and washed with water. The organic phase was dried and concentrated in vacuo to give a light brown gum that was used without further purification. APCI-MS: m/z 494.1 [MH⁺].

### Intermediate 5

### S-(4-Hydroxybut-2-yn-1-yl)(4aS,4bR,6aS,6bS,9aR,10aS,10bS,12S)-4b,12-Difluoro-4a,6a,8,8-tetramethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carbothioate

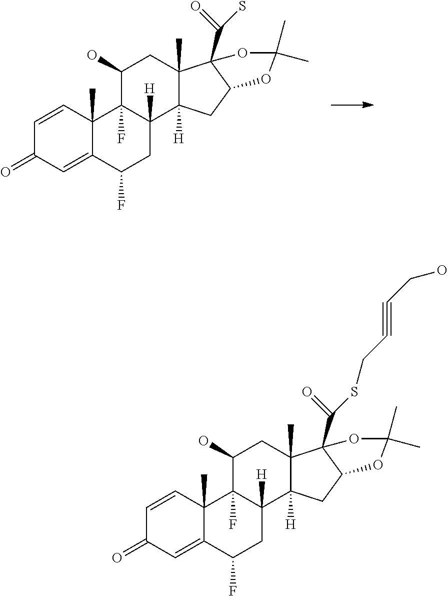

The same preparation was followed as for Intermediate 4, using 1-chloro-4-hydroxybut-2-yne. APCI-MS: m/z 523.1 [MH⁺].

### Intermediate 6

### S-(Fluoromethyl)(4aS,4bR,6aS,6bS,9aR,10aS,10bS,12S)-4b,12-Difluoro-4a,6a,8,8-tetramethyl-2-oxo-2,4a,4b,5,6,6a,9a,10,10a,10,11,12-dodecahydro-6bH-naphtho[2′,1′:4,5]-indeno[1,2-d][1,3]dioxole-6b-carbothioate

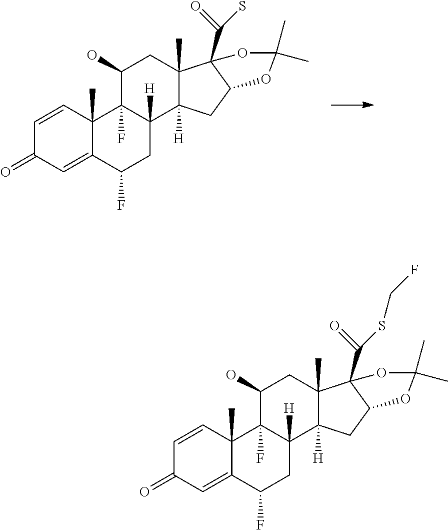

The same preparation was followed as for Intermediate 4, using 1-bromo-1-fluoromethane. APCI-MS: m/z 487.1 [MH⁺].

### Intermediate 7

### (6α,11β,16α,17α)-6,9-Difluoro-11,16,17-trihydroxy-3-oxoandrosta-1,4-diene-17-carboxylic acid

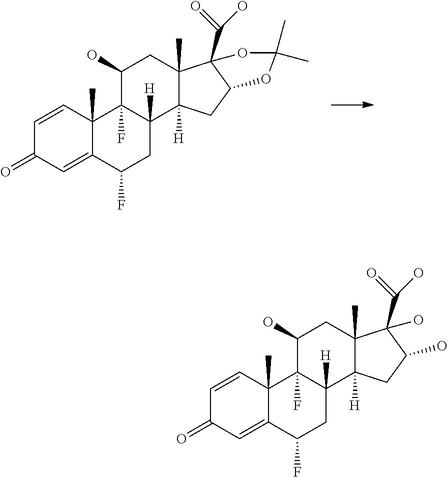

A solution of Intermediate 1 (750 mg, 1.71 mmol) in formic acid (10 mL) was stirred at 80° C. under an atmosphere of nitrogen for 2 hrs to give a clear solution. The reaction mixture was concentrated under reduced pressure, the residue was dissolved in formic acid (10 mL) and the obtained solution was stirred at 42° C. overnight. The mixture was concentrated under reduced pressure, the obtained residue was redissolved in toluene, and the solvent was removed under reduced pressure again. The obtained residue was redissolved in dioxan (10 mL) and the pH was adjusted to 10-11 using aq. NaOH (2M). The obtained mixture was stirred for 1 h and the organic solvent was removed under reduced pressure. Water (10 mL) was added to the wet residue and the pH was adjusted to 7 with few drops of conc. HCl. The aq. phase was washed with EtOAc, acidified with conc. HCl to pH 2 and the resulting white precipitate was filtered, washed with water and dried on the sinter to give 512 mg (75%) of the desired compound as a white powder. The product was used as such in the next step without further purification. APCI-MS: m/z 399 [MH⁺].

### Intermediate 8

### (4aS,4bR,5S,6aS,6bS,9aR,10aS,10bS,12S)-4b,12-Difluoro-5-hydroxy-4a,6a-dimethyl-2-oxo-8-(2-thienyl)-2,4a,4b,5,6,6a,9a,10,10a,10,11,12-dodecahydro-6bH-naphtho[2′,1′:4,5]indeno[1,2-d][1,3]dioxole-6b-carboxylic acid

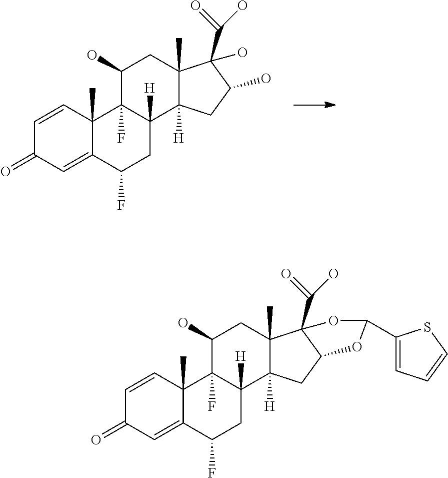

In a round bottomed flask intermediate 7 (500 mg, 1.26 mmol) was suspended in THF (5 ml) and thiophene-2-carbaldehyde (565 mg, 5.04 mmol) was added followed by perchloric acid (0.12 mmol, 0.026 mL). Stirring was continued at RT for 2 hours and resulted in a clear solution. Triethylamine was added to neutralise the acid. The solvent was evaporated, the residue dissolved in MeCN and precipitated by addition of water. The precipitate obtained was filtered, washed with water and dried in vacuo to yield 550 mg (89%) of a solid which was used directly in the next step without further purification. APCI-MS: m/z 493.1 [MH⁺].

### Intermediate 8a

### (4aS,4bR,5S,6aS,6bS,9aR,10aS,10bS,12S)-4b,12-Difluoro-5-hydroxy-4a,6a-dimethyl-8-(5-methyl-2-furyl)-2-oxo-2,4a,4b,5,6,6a,9a,10,10a,10,11,12-dodecahydro-6bH-naphtho[2′,1′:4,5]indeno[1,2-d][1,3]dioxole-6b-carboxylic acid

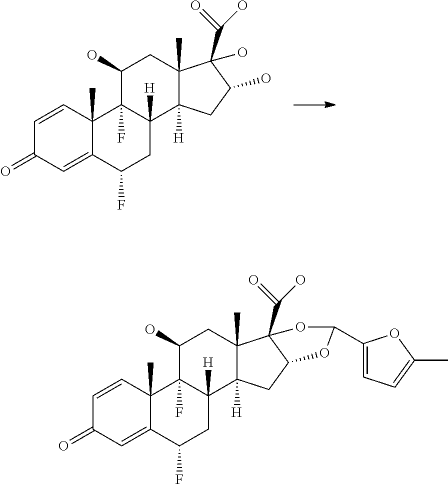

In a round bottomed flask intermediate 7 (500 mg, 1.26 mmol) was suspended in THF and 5-methylfuran-2-carbaldehyde (550 mg, 5.04 mmol) was added followed by perchloric acid (0.026 mL, 0.12 mmol). Stirring at RT was continued for 2 hours. The reaction mixture was neutralised by addition of triethylamine. The solvent was removed under reduced pressure, the obtained residue was dissolved in 2N aq. NaOH and the resulting solution was washed with diethylether several times. After acidification with 6N HCl the obtained precipitate was filtered, washed with water and dried in vacuo. The obtained brown solid was washed with DCM several times to afford the desired product (550 mg; 89%) as a yellowish solid. The crude product was used directly in the next step without further purification. APCI-MS: m/z 491.0 [MH⁺].

### Intermediate 8b

### (4aS,4bR,5S,6aS,6bS,9aR,10aS,10bS,12S)-8-(2,4-Difluorophenyl)-4b,12-difluoro-5-hydroxy-4a,6a-dimethyl-2-oxo-2,4a,4b,5,6,6a,9a,10,10a,10,11,12-dodecahydro-6bH-naphtho[2′,1′:4,5]indeno[1,2-d][1,3]dioxole-6b-carboxylic acid

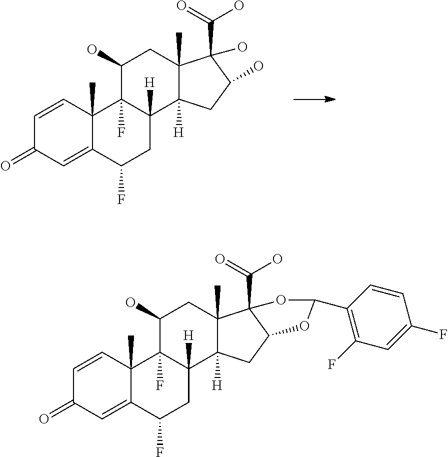

In a round bottomed flask intermediate 7 (500 mg, 1.26 mmol) was suspended in THF (5 mL) and 2,4-difluoro-benzaldehyde (716 mg, 5.04 mmol) was added followed by perchloric acid (0.12 mmol, 0.026 mL). Stirring at RT was continued for 2 hours to obtain a clear solution. Triethylamine was added to neutralise the acid and the solvent was removed under reduced pressure. The obtained residue was dissolved in 2N NaOH and the solution was washed with diethyl ether several times. Acidification with 6N HCl yielded a precipitate which was filtered, washed with water and dried in vacuo to result in 550 mg (84%) of the desired product as a white solid which was used directly in the next step without further purification. APCI-MS: m/z 523.2 [MH⁺].

### Intermediate 9

### S-{[(4aS,4bR,5S,6aS,6bS,9aR,10aS,10bS,12S)-4b,12-Difluoro-5-hydroxy-4a,6a-dimethyl-2-oxo-8-(2-thienyl)-2,4a,4b,5,6,6a,9a,10,10a,10b,11,12-dodecahydro-6bH-naphtho[2′,1′:4,5]indeno[1,2-d][1,3]dioxol-6b-yl]carbonyl}dimethylthiocarbamate

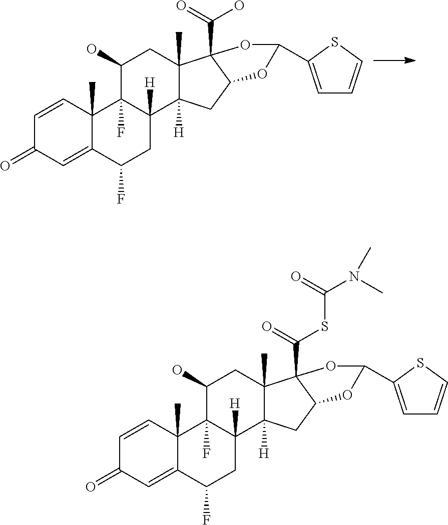

In a round bottomed flask was dissolved Intermediate 8 (500 mg, 1.02 mmol), N,N-dimethylcarbamothioic chloride (378 mg 3.06 mmol), triethylamine (0.426 mL, 3.06 mmol) and sodium iodide (0.20 mmol, 30 mg) in acetone/water (15 mL, 2:1) and the reaction mixture was stirred at room temperature for 4 hours. N,N-dimethyl acetamide (1 mL) was added and the reaction mixture was stirred for a further 30 min. The precipitate obtained was filtered, washed with water and dried in vacuo to give 450 mg of the desired compound (76%) as an off white solid. The crude product was used directly in the next step without further purification. APCI-MS: m/z 580.2 [MH⁺].

### Intermediate 9a

### S-{[(4aS,4bR,5S,6aS,6bS,9aR,10aS,10bS,12S)-4b,12-Difluoro-5-hydroxy-4a,6a-dimethyl-8-(5-methyl-2-furyl)-2-oxo-2,4a,4b,5,6,6a,9a,10,10a,10b,11,12-dodecahydro-6bH-naphtho[2′,1′:4,5]indeno[1,2-d][1,3]dioxol-6b-yl]carbonyl}dimethylthiocarbamate

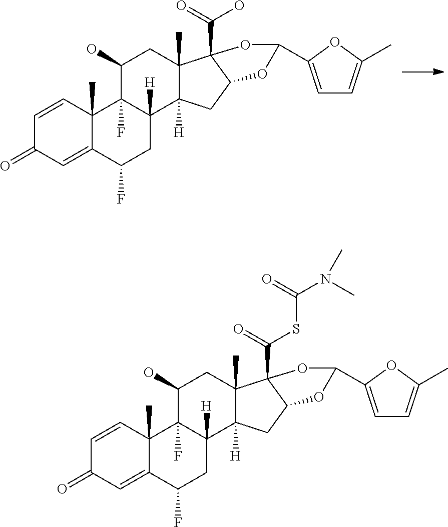

In a round bottomed flask was dissolved intermediate 8a (500 mg, 1.02 mmol), N,N-dimethylcarbamothioic chloride (378 mg 3.06 mmol), triethylamine (0.426 mL, 3.06 mmol) and sodium iodide (0.20 mmol, 30 mg) in acetone-water (15 mL, 2:1) and the reaction mixture was stirred at room temperature for 4 hours. N,N-dimethyl acetamide (1 mL) was added and the reaction mixture was stirred for a further 30 min. The precipitate obtained was filtered, washed with water and dried in vacuo to give 460 mg (78%) of the desired product a white solid which was taken on as such in the next step without further purification. APCI-MS: m/z 578.0 [MH⁺].

### Intermediate 9b

### S-{[(4aS,4bR,5S,6aS,6bS,9aR,10aS,10bS,12S)-8-(2,4-Difluorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxol-6b-yl]carbonyl}dimethylthiocarbamate

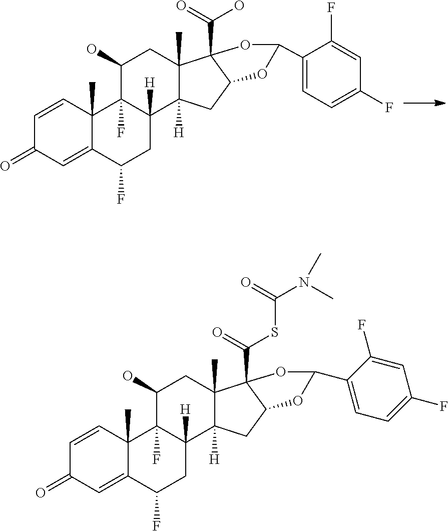

In a round bottomed flask was dissolved intermediate 8b (522 mg, 1.0 mmol), N,N-dimethylcarbamothioic chloride (378 mg 3.06 mmol), triethylamine (0.426 mL, 3.06 mmol) and sodium iodide (0.20 mmol, 30 mg) in acetone-water (15 mL, 2:1) and the reaction mixture was stirred at room temperature for 4 hours. N,N-dimethyl acetamide (1 mL) was added and the reaction mixture was stirred for a further 30 min. The precipitate obtained was filtered, washed with water and dried in vacuo to give 460 mg (75%) of the desired product as a white solid which was taken on as such in the next step without further purification. APCI-MS: m/z 610.0 [MH⁺].

### Intermediate 10

### (8S,9S,10R,11S,13S,14S,16R,17S)-11,16,17-Trihydroxy-10,13-dimethyl-3-oxo-6,7,8,9,10,11,12,13,14,15,16,17-dodecahydro-3H-cyclopenta[a]phenanthrene-17-carboxylic acid

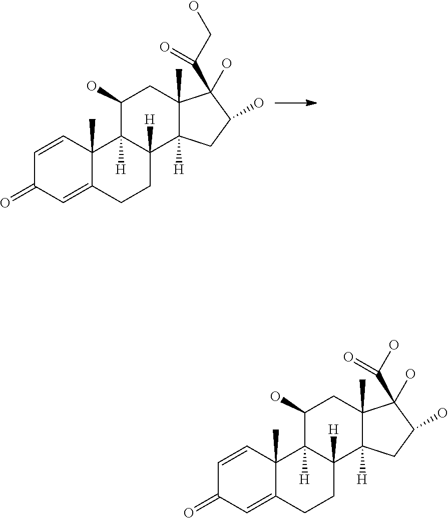

To a stirred solution of (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (2.0 g, 5.31 mmol) in DMF (30 ml) was added potassium superoxide (1.51 g, 21.3 mmol) followed by 1,4,7,10,13,16-hexaoxacyclooctadecane (2.64 g, 5.31 mmol). The reaction mixture was cooled using a water bath, so that the temperature was kept below 28° C. The mixture was subsequently stirred at r.t. for 5 h, then poured slowly into water (300 ml). The water solution was extracted with ethyl acetate (100 ml) and discarded. After acidification with conc. HCl the aqueous phase was extracted with ethyl acetate (3 times 100 ml) and the combined organic extracts were washed with brine. Evaporation of solvent afforded colourless oil, which was redissolved in aq. NaOH solution (2M, 100 ml). The alkaline solution washed with ethyl acetate (2×50 ml) and discarded. After acidification with conc. HCl the aqueous phase was extracted with ethyl acetate (3×50 ml) and the combined extracts were dried with anhydrous Na₂SO₄. Evaporation of solvent afforded the target compound as a colourless solid, 1.25 g (65%). APCI-MS: m/z 363 [MH⁺].

### Intermediate 11

### S-{[(4aR,4bS,5S,6aS,6bS,9aR,10aS,10bS)-8-(2-Fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxol-6b-yl]carbonyl}dimethylthiocarbamate

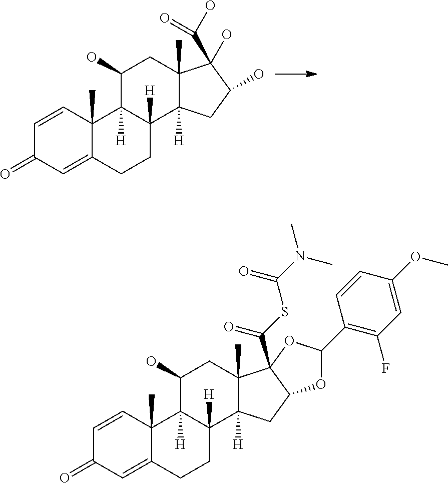

To a stirred solution of intermediate 10 (300 mg, 0.83 mmol) in THF (5 mL) was added 2-fluoro-4-methoxybenzaldehyde (638 mg, 4.14 mmol) followed by several drops of perchloric acid (70%). The mixture was stirred at RT overnight, then triethylamine (100 μl) was added to neutralize the acid, and the solvent was removed in vacuo. The residue was redissolved in aq. NaOH (2 M, 20 ml), forming an emulsion, which was washed with diethyl ether (3×20 ml). The aqueous layer was acidified with conc. HCl, and extracted with ethyl acetate (3×20 ml). The combined organic extracts were dried with anhydrous Na₂SO₄. The solvent was removed in vacuo to afford (4aR,4bS,5S,6aS,6bS,9aR,10aS,10bS)-8-(2-fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carboxylic acid as a colourless oil (286 mg, 0.57 mmol) which was dissolved in acetone (10 ml). To the stirred solution was added N,N-dimethylcarbamothioic chloride (213 mg, 1.72 mmol) followed by triethylamine (0.24 ml, 1.72 mmol). Then sodium iodide (17 mg, 0.11 mmol) was added, followed by water (0.1 ml). The mixture was stirred for 24 hrs at RT, then concentrated in vacuo, diluted with DMA (1 ml), and poured into cold water (30 ml). The precipitate was collected by filtration, washed with water on the filter, and dried to afford 181 mg (37%) of the desired product as yellow solid. APCI-MS: m/z 586 [MH⁺].

### Intermediate 12

### S-{[(4aR,4bS,5S,6aS,6bS,9aR,10aS,10bS)-8-(2,4-Dimethylphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxol-6b-yl]carbonyl}dimethylthiocarbamate

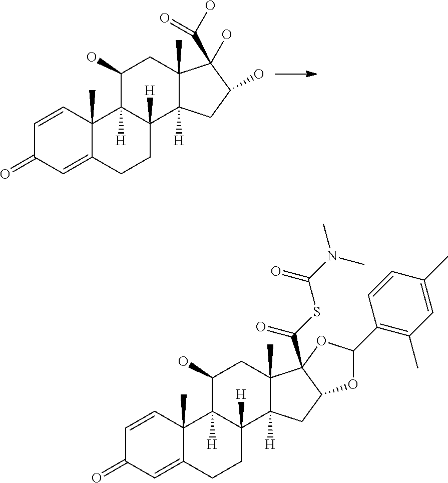

Prepared from Intermediate 10 and 2,4-dimethylbenzaldehyde following the procedure described for Intermediate 11. APCI-MS: m/z 566 [MH⁺].

### Intermediate 13

### S-({(4aR,4bS,5S,6aS,6bS,9aR,10aS,10bS)-5-Hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxol-6b-yl}carbonyl)dimethylthiocarbamate

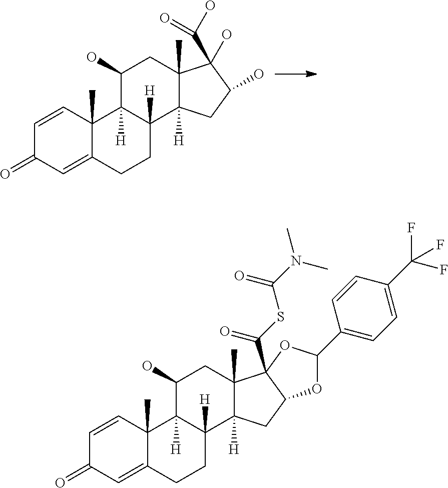

Prepared from Intermediate 10 and 4-trifluoromethylbenzaldehyde following the procedure described for Intermediate 11. APCI-MS: m/z 606 [MH⁺].

### Intermediate 14

### S-{[(4aR,4bS,5S,6aS,6bS,9aR,10aS,10bS)-8-(2-Fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxol-6b-yl]carbonyl}dimethylthiocarbamate

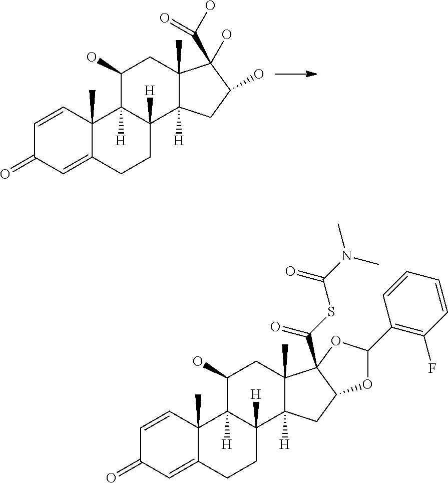

Prepared from Intermediate 10 and 2-fluorobenzaldehyde following the procedure described for Intermediate 11. APCI-MS: m/z 556 [MH⁺].

### Intermediate 15

### (4aR,4bS,5S,6aS,6bS,9aR,10aS,10bS)-5-Hydroxy-4a,6a,8,8-tetramethyl-2-oxo-2,4a,4b,5,6,6a,9a,10,10a,10b,11,12-dodecahydro-6bH-naphtho[2′,1′:4,5]indeno[1,2-d][1,3]dioxole-6b-carboxylic acid

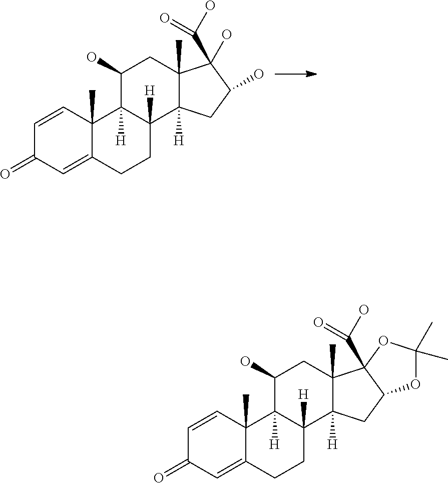

To a stirred solution of intermediate 10 (3.62 g, 10 mmol) in acetone (300 ml) was added perchloric acid (70% wt., 100 μl). The mixture was stirred at RT for 3 hrs, then the mixture was poured into water (600 ml) and extracted with ethyl acetate (4×200 ml). The combined organic extracts were dried over Na₂SO₄ and the filtered solvent was evaporated in vacuo to afford the target compound as a white solid, 2.12 g (53%). APCI-MS: m/z 403 [MH⁺].

### Intermediate 16

### S-{[(4aR,4bS,5S,6aS,6bS,9aR,10aS,10bS)-5-Hydroxy-4a,6a,8,8-tetramethyl-2-oxo-2,4a,4b,5,6,6a,9a,10,10a,10b,11,12-dodecahydro-6bH-naphtho[2′,1′:4,5]indeno[1,2-d][1,3]dioxol-6b-yl]carbonyl}dimethylthiocarbamate

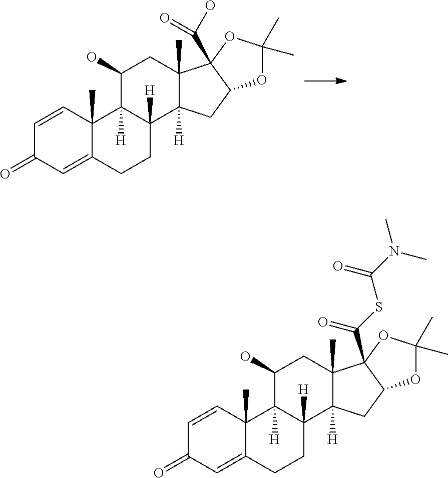

To a stirred solution of Intermediate 15 (2.12 g, 5.3 mmol) in acetone (50 ml) were added N,N-dimethylcarbamothioic chloride (1.95 g, 15.8 mmol), triethylamine (2.19 ml, 15.8 mmol), sodium iodide (158 mg, 1.05 mmol) and water (0.1 ml). Stirring was continued for 24 h at RT. Then another portion of N,N-dimethylcarbamothioic chloride (0.98 g, 7.9 mmol) and triethylamine (1.1 ml, 7.9 mmol) were added and stirring was continued for another 24 hrs. The mixture was concentrated in vacuo, the obtained residue was diluted with N,N-dimethylacetamide (10 ml) and poured into cold water (100 ml). The resulting precipitate was collected by filtration, washed with water on the sinter and dried to yield 2.24 g of the target compound (87%). APCI-MS: m/z 490 [MH⁺].

### Intermediate 17

### (4aR,4bS,5S,6aS,6bS,9aR,10aS,10bS)-5-Hydroxy-4a,6a,8,8-tetramethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioic S-acid

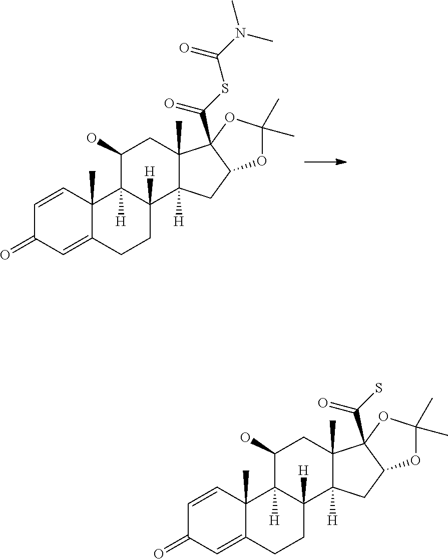

Prepared from Intermediate 16 following the procedure described for Intermediate 3. APCI-MS: m/z 419 [MH⁺].

### Intermediate 18

### S-(Fluoromethyl)(4aR,4bS,5S,6aS,6bS,9aR,10aS,10bS)-5-hydroxy-4a,6a,8,8-tetramethyl-2-oxo-2,4a,4b,5,6,6a,9a,10,10a,10,11,12-dodecahydro-6bH-naphtho[2′,1′:4,5]indeno[1,2-d][1,3]dioxole-6b-carbothioate

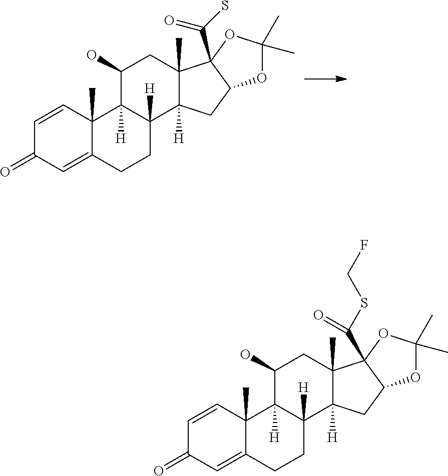

Prepared from Intermediate 17, using bromofluoromethane, following the procedure described for Intermediate 4. APCI-MS: m/z 451 [MH⁺].

### Example 1

### S-(Cyanomethyl)(4aS,4bR,6aS,6bS,9aR,10aS,10bS,12S)-4b,12-difluoro-8-(2-furyl)-4a,6a-dimethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carbothioate

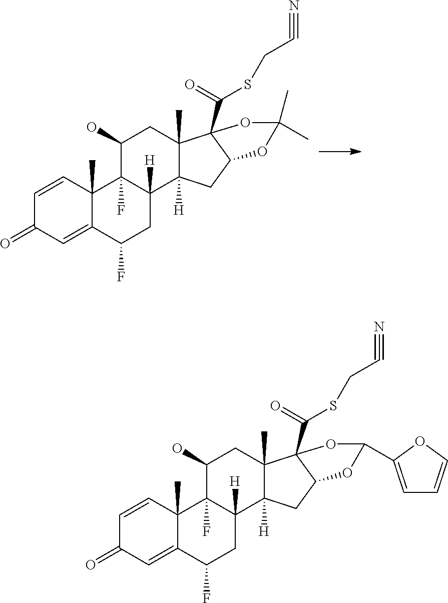

To a solution of intermediate 4 (604 mg, 1.22 mmol) in dichloromethane (2.0 ml) was added 2-furaldehyde (0.140 ml, 1.69 mmol), followed by 1-butyl-3-methylimidazolium hexafluorophosphate (1.0 ml, 4.86 mmol) and perchloric acid (300 ul, 4.96 mmol). The mixture was stirred at 28° C. for 20 minutes. Additional aldehyde (0.130 ml, 1.57 mmol) was added and after 2 hrs the mixture was poured into a cold solution of saturated aqueous sodium bicarbonate and extracted with dichloromethane. The organic phase was separated, dried and concentrated in vacuo. The crude material was purified on HPLC water/CH₃CN 0.1% TFA (gradient of 20% to 85% within 40 min.) to give 50 mg (0.09 mmol, 8%) as a 80:16 mixture of the 8-(R)- and 8-(S)-diastereomers, according to ¹H-NMR spectroscopy.

¹H-NMR (400 MHz, DMSO) δ 7.71 (1H, d, R), 7.59 (1H, d, S), 7.26 (1H, m), 6.65 (1H, d), 6.47 (1H, m, R), 6.42 (1H, d, S), 6.35 (1H, m, S), 6.30 (1H, d), 6.28 (1H, d), 6.26 (1H, s, S), 6.17 (1H, s), 5.74 (1H, s, R), 5.69-5.65 (1H, m), 5.57 (3H, m), 5.31 (1H, d, S), 4.96 (1H, d, R), the remaining signals appear between: 4.41-0.96 ppm; APCI-MS: m/z 532.1 [MH⁺].

### Example 2

### S-(4-Hydroxybut-2-yn-1-yl)(4aS,4bR,6aS,6bS,9aR,10aS,10bS,12S)-4b,12-difluoro-8-(2-furyl)-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

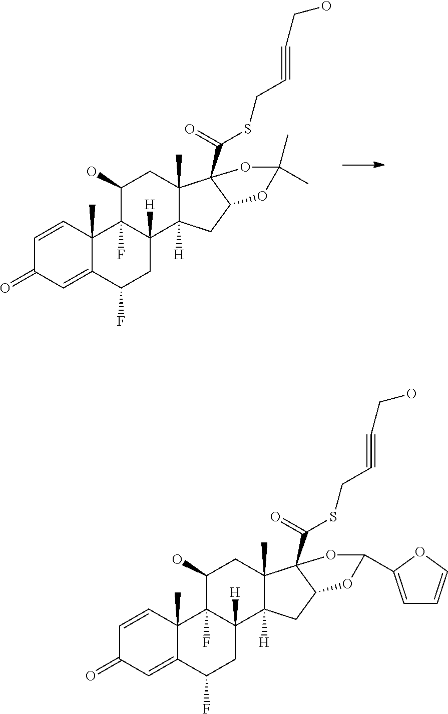

The same preparation was followed as in Example 1, using Intermediate 5. The product was isolated as 76:24—mixture of 8-(R)- and 8-(S)-diastereomers according to ¹H-NMR spectroscopy.

¹H-NMR (400 MHz, DMSO) δ 7.69 (1H, d, R), 7.59 (1H, d, S), 7.26 (1H, m), 6.60 (1H, d), 6.63 (1H, m, R), 6.42 (1H, d, S), 6.35 (1H, m, S), 6.30 (1H, d), 6.28 (1H, d), 6.26 (1H, s, S), 6.18 (1H, s), 5.70 (1H, s, R), 5.68-5.54 (1H, m) 5.48 (1H, d, S), 5.30 (1H, d, R), the remaining signals appear between: 4.24-0.93 ppm; APCI-MS: m/z 561.1 [MH⁺]

### Example 3

### S-(Fluoromethyl)(4aS,4bR,6aS,6bS,9aR,10aS,10bS,12S)-4b,12-difluoro-8-(2-furyl)-4a,6a-dimethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carbothioate

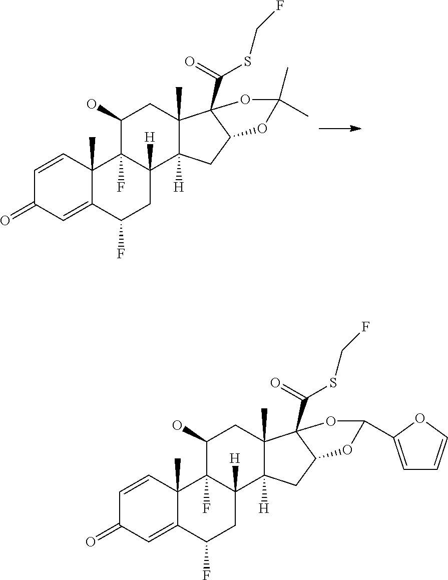

The same preparation was followed as in Example 1, using Intermediate 6. The product was isolated as 72:28—mixture of 8-(R)- and 8-(S)-diastereomers according to ¹H-NMR spectroscopy.

¹H-NMR (400 MHz, CD₃CN) δ 7.54 (1H, d, R), 7.44 (1H, d, S), 7.20-7.18 (1H, m), 6.60 (1H, d), 6.44 (1H, m, R), 6.42 (1H, d, S), 6.35 (1H, m, S), 6.30 (1H, d), 6.28 (1H, d), 6.26 (1H, s, S), 6.24 (1H, s), 5.89-5.82 (1H, m), 5.71 (1H, s, R), 5.57-5.53 (1H, m), 5.36 (1H, d, S), 5.02 (1H, d, R), the remaining signals appear between: 2.72-0.99 ppm; APCI-MS: m/z 525.1 [MH⁺].

### Example 4

### S-Methyl (4aS,4bR,5S,6aS,6bS,8S,9aR,10aS,10bS,12S)-4b,12-difluoro-5-hydroxy-4a,6a-dimethyl-2-oxo-8-(2-thienyl)-2,4a,4b,5,6,6a,9a,10,10a,10,11,12-dodecahydro-6bH-naphtho[2′,1′:4,5]indeno[1,2-d][1,3]dioxole-6b-carbothioate

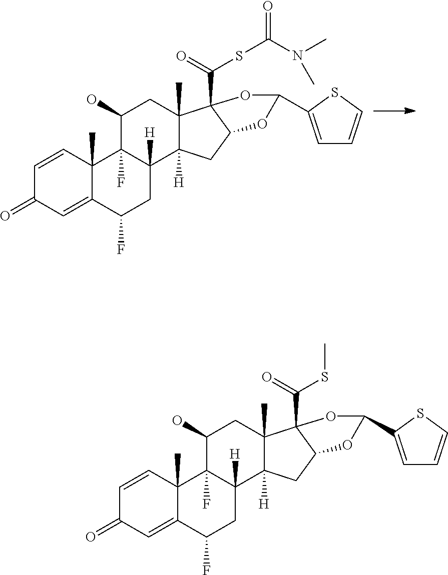

In a round bottomed flask was dissolved intermediate 9 (400 mg, 0.69 mmol) and potassium carbonate (238 mg, 1.72 mmol) in methanol (4 mL) and stirring was continued at room temperature for 30 min. Water (1 mL) was added to the reaction mixture and the aq. phase was washed with toluene. The aq. phase was acidified with 2N HCl to pH 1 and the resulting precipitate was filtered, washed with water and dried in vacuo to afford the respective thio acid as a minor product and methyl ester as a major bi-product. The crude mixture (50 mg, appr. 0.10 mmol) was dissolved in acetonitrile (4 mL) and potassium carbonate (21 mg, 0.15 mmol) was added at room temperature followed by iodomethane (13.95 mg, 0.10 mmol). Stirring was continued for 1 hour at the same temperature and the reaction mixture was partitioned between ethylacetate and water. The organic phase was dried (Na₂SO₄), filtered and concentrated in vacuo. The crude mixture was purified by HPLC using MeCN—H₂O (30-100% MeCN in 20 min) to give the desired product (5 mg, 10%) as a colourless solid.

¹H NMR (400 MHz, CD₃CN) δ 7.48 (1H, d), 7.23-7.20 (1H, m), 7.18 (1H, d), 7.03-7.00 (1H, m), 6.29 (1H, d), 6.25 (1H, s), 5.93 (1H, s), 5.60-5.54 (1H, m), 5.48-5.42 (1H, m), 5.00-4.98 (1H, m), 4.36-4.30 (1H, m), 3.42-3.36 (1H, m), 2.72-2.57 (2H, m), 2.45-2.32 (2H, m), 2.32 (3H, s), 1.82-1.74 (2H, m), 1.68-1.60 (2H, m), 1.52 (3H, s), 0.99 (3H, s); APCI-MS: m/z 523.1 [MH⁺].

### Example 5

### S-Methyl (4aS,4bR,5S,6aS,6bS,9aR,10aS,10bS,12S)-8-(2,4-difluorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

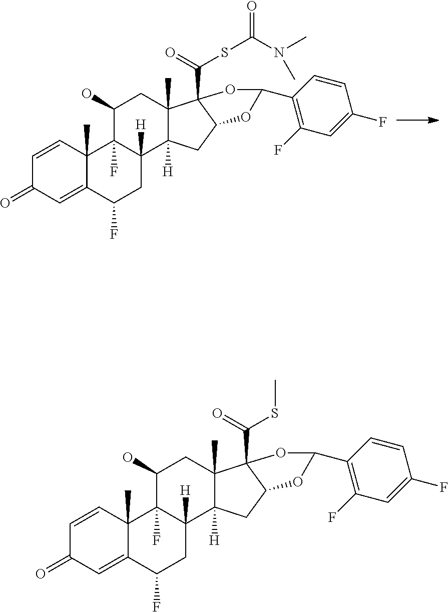

The same preparation was followed as in Example 4, using Intermediate 9b and iodomethane as alkylating reagent. The product was isolated as 1:1—mixture of 8-(R)- and 8-(S)-diastereomers according to ¹H-NMR spectroscopy.

¹H-NMR (400 MHz, CD₃CN) δ 7.61-7.67 (1H, d, R), 7.48-7.42 (1H, m, S) 7.21-7.18 (2H, m), 7.05-6.90 (2H, m), 6.46 (1H, s), 6.31-6.24 (2H, m), 5.84 (1H, s), 5.59-5.54 (1H, m), 5.47-5.43 (1H, m), 5.41 (1H, d), 5.05 (1H, m), 4.36-4.31 (1H, m), 2.75-2.59 (2H, m), 2.38 (3H, s), 2.09-2.18 (2H, m), 1.62-1.87 (2H, m), 1.51 (3H, s), 1.02 (2H, s, R), 1.01 (2H, s, S); APCI-MS: m/z 553.2 [MH⁺].

### Example 6

### S-(Fluoromethyl)(4aS,4bR,5S,6aS,6bS,9aR,10aS,10bS,12S)-8-(2,4-difluorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

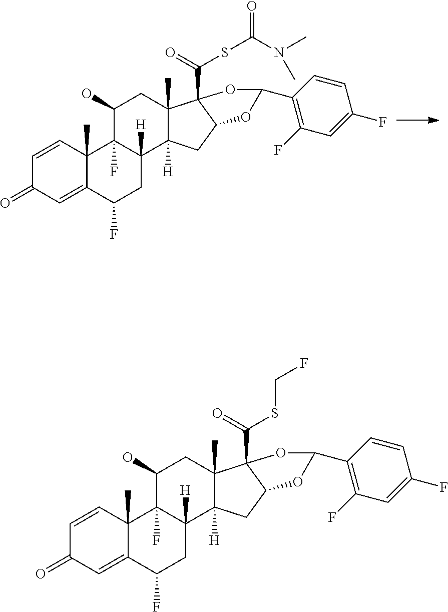

The same preparation was followed as in Example 4, using Intermediate 9b and bromofluoro methane as alkylating reagent. The product was isolated as 2:1 mixture of 8-(R)- and 8-(S)-diastereomers according to ¹H-NMR spectroscopy.

¹H-NMR (400 MHz, CD₃CN) δ 7.66-7.58 (1H, d), 7.44-7.38 (1H, m), 7.20-7.17 (1H, m), 7.03-6.90 (1H, m), 6.46 (1H, s), 6.31-6.23 (1H, m), 6.01 (1H, d), 5.90-5.80 (1H, m), 5.75-5.68 (1H, m), 5.59-5.53 (1H, m), 5.47-5.41 (1H, m), 5.05 (1H, d), 4.35-4.30 (1H, m), 2.75-2.56 (1H, m), 2.39-2.23 (2H, m), 2.18-2.08 (1H, m), 1.92-1.77 (2H, m), 1.76-1.61 (1H, m), 1.51 (3H, s), 1.02 (3H, s); APCI-MS: m/z 571.2 [MH⁺].

### Example 7

### S-(Fluoromethyl)(4aS,4bR,5S,6aS,6bS,8S,9aR,10aS,10bS,12S)-4b,12-difluoro-5-hydroxy-4a,6a-dimethyl-8-(5-methyl-2-furyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

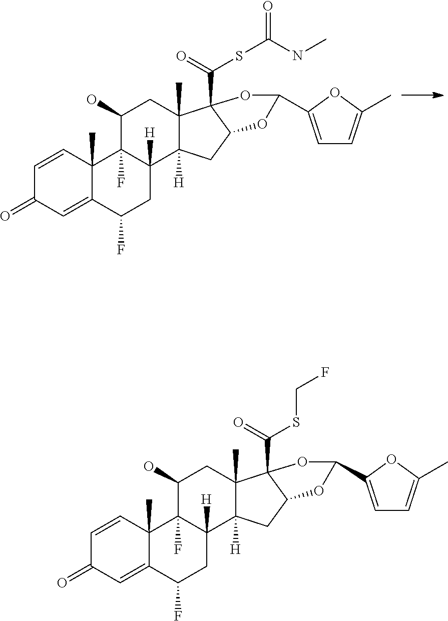

The same preparation was followed as in Example 4, using Intermediate 9a and iodomethane as alkylating reagent.

¹H-NMR (400 MHz, CD₃CN) δ 7.20-7.17 (1H, d), 6.60 (1H, d), 6.30 (1H, d), 6.28 (1H, d), 6.27 (1H, s), 6.18 (1H, s), 5.93-5.92 (1H, m), 5.75-5.72 (1H, m), 5.58-5.53 (1H, m), 5.46-5.42 (1H, m), 5.34 (1H, d), 4.33 (1H, m), 3.42 (1H, m), 2.46 (2H, m), 2.39-2.23 (2H, m), 1.92-1.67 (2H, m), 1.51 (3H, s), 0.99 (3H, s); APCI-MS: m/z 539.3 [MH⁺].

### Example 8

### S-Methyl (4aS,4bR,5S,6aS,6bS,9aR,10aS,10bS,12S)-4b,12-difluoro-5-hydroxy-4a,6a-dimethyl-8-(5-methyl-2-furyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

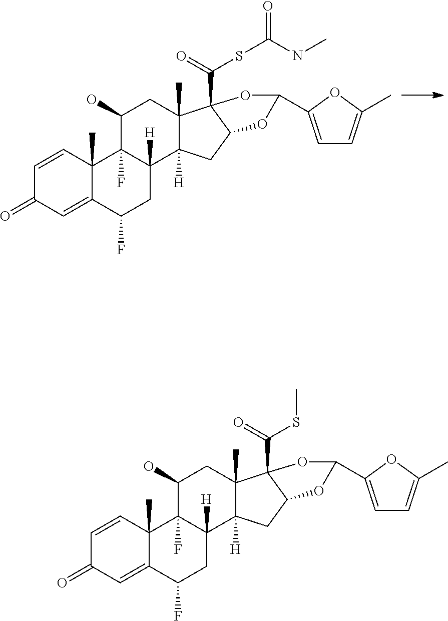

The same preparation was followed as in Example 4, using Intermediate 9a and bromofluoro methane as alkylating reagent. The compound was isolated as a 3:1 mixture of 8-(R)- and 8-(S)-diastereomers according to ¹H-NMR spectroscopy.

¹H-NMR (400 MHz, CD₃CN) δ 7.20-7.18 (1H, m), 6.43 (1H, d), 6.30-6.25 (2H, m), 6.24 (1H, s), 6.14 (1H, s), 5.93 (1H, m), 5.63 (1H, m), 5.56-5.53 (1H, m), 5.44-5.42 (1H, m), 5.32 (1H, d), 4.99 (1H, m), 4.33 (1H, m), 3.42 (1H, m), remaining proton signals appear between: 2.68-0.96 ppm; APCI-MS: m/z 521.3 [MH⁺].

### Example 9

### S-(Cyanomethyl)(4aS,4bR,5S,6aS,6bS,9aR,10aS,10bS,12S)-4b,12-difluoro-5-hydroxy-4a,6a-dimethyl-2-oxo-8-(2-thienyl)-2,4a,4b,5,6,6a,9a,10,10a,10b,11,12-dodecahydro-6bH-naphtho[2′,1′:4,5]indeno[1,2-d][1,3]dioxole-6b-carbothioate

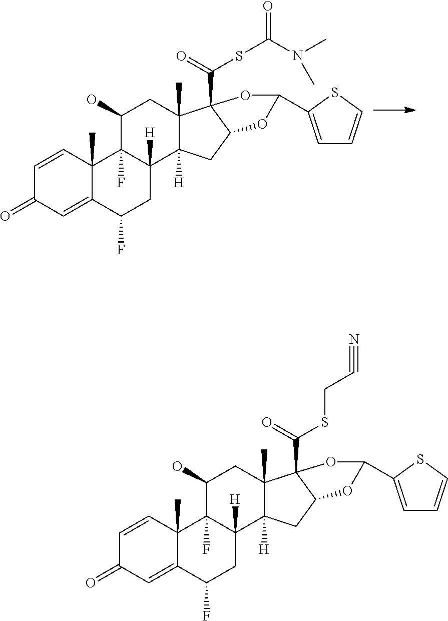

In a round bottomed flask was dissolved Intermediate 9 (30 mg, 0.05 mmol) in N,N-dimethyl acetamide (1 mL) and NaSH (28 mg, 0.5 mmol) was added at room temperature. The reaction mixture was stirred for 30 min. at the same temperature and 1 N HCl (1 mL) was subsequently added to the reaction mixture. The desired thio-acid was extracted with ethyl acetate, the organic phase was washed with water, dried over sodium sulfate, filtered and evaporated to yield 20 mg (76%) of an off white solid. The crude thio-acid (20 mg, 0.04 mmol) was dissolved in acetonitrile (1 mL) and potassium carbonate (11 mg, 0.08 mmol) followed by bromoacetonitrile (5 mg, 0.08 mmol) was added at room temperature. The reaction mixture was stirred for 30 min. at the same temperature and the solvent was removed in vacuo. The product was extracted with ethyl acetate, the organic phase was washed with water, dried over sodium sulfate, filtered and the solvent was removed in vacuo. The crude product was purified by HPLC (MeCN-water 40-100% in 20 min.) to yield 15 mg (70%) of the desired product as a colourless solid. The product was isolated as a 20:1 mixture of 8-(R)- and 8-(S)-diastereomers according to ¹H-NMR spectroscopy.

¹H-NMR (400 MHz, CD₃CN) δ 7.50 (1H, d), 7.25-7.23 (1H, m), 7.20-7.17 (1H, m), 7.03-7.01 (1H, m), 6.30-6.25 (2H, m), 5.98 (1H, m), 5.90-5.85 (1H, m), 5.59-5.55 (1H, m), 5.47-5.42 (1H, m), 5.00-4.99 (1H, m), 4.35-4.32 (1H, m), 3.81-3.70 (1H, m), 3.48-3.46 (1H, m), 2.72-2.64 (1H, m), 2.76-2.61 (1H, m), 2.43-2.33 (1H, m), 2.14 (1H, s), 1.84-1.61 (1H, m), 1.52 (3H, s), 1.04 (3H, s); APCI-MS: m/z 548.2 [MH⁺].

### Example 10

### S-(Cyanomethyl)(4aS,4bR,5S,6aS,6bS,9aR,10aS,10bS,12S)-4b,12-difluoro-5-hydroxy-4a,6a-dimethyl-8-(5-methyl-2-furyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

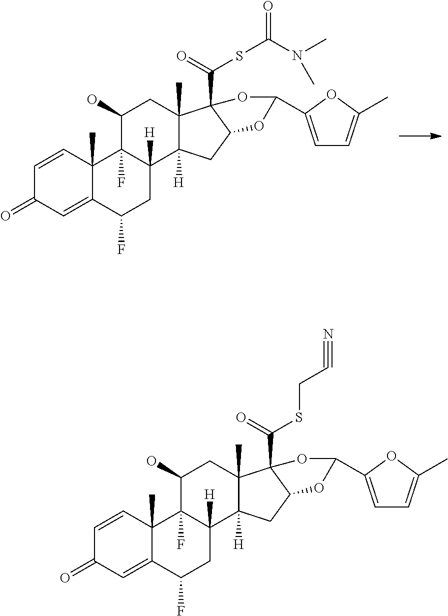

The same preparation was followed as in Example 9, using Intermediate 9a and bromo acetonitrile as an alkylating agent. The product was isolated as a 3:1 mixture of 8-(R)- and 8-(S)-diastereomers according to ¹H-NMR spectroscopy.

¹H-NMR (400 MHz, CD₃CN) δ 7.02 (1H, m), 6.65 (1H, d), 6.46 (1H, m), 6.30-6.18 (2H, m, S), 6.02 (1H, m), 5.69 (1H, s), 5.59-5.54 (1H, m), 5.47-5.41 (1H, m), 5.00 (1H, m); 4.35-4.31 (1H, m), 3.81 (1H, m), 3.46 (1H, m), remaining proton signals appear between 2.73-1.02 ppm; APCI-MS: m/z 546.2 [MH⁺].

### Example 11

### S-(Fluoromethyl)(4aS,4bR,5S,6aS,6bS,9aR,10aS,10bS,12S)-8-(5-chloro-2-furyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

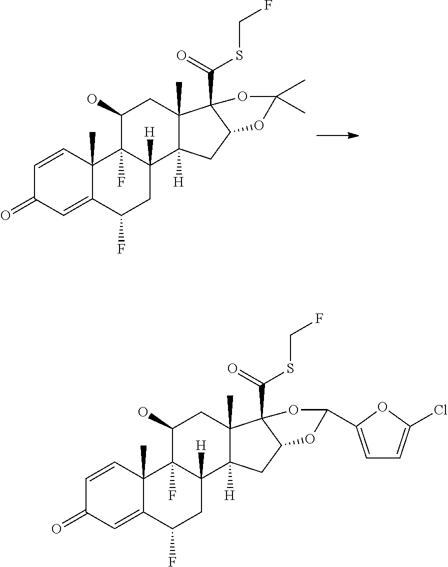

In a 4 mL vial intermediate 4 (50 mg, 0.1 mmol) was suspended in 2 mL of DCM. Perchloric acid (0.050 mL, 0.23 mmol) was added, the vial was capped and shaken for 5 min. 5-Chlorofuran carbaldehyde (19 mg, 0.15 mmol) was added to the mixture and the vial was shaken for another 15 min. Saturated aq. NaHCO₃ was added to neutralise the acid and the product was extracted with DCM. The organic phase was washed with water, dried over sodium sulfate, filtered and the solvent was removed in vacuo. The crude product was purified by HPLC (MeCN-water 40-100% MeCN in 20 min) to afford 10 mg (17%) of the desired product as an off-white solid. The product was isolated as 2:1 mixture of 8-(R)- and 8-(S)-diastereomers according to ¹H-NMR spectroscopy.

¹H-NMR (400 MHz, CD₃CN) δ 7.20-7.17 (1H, m), 6.63 (1H, d), 6.43 (1H, m), 6.42 (1H, d), 6.35 (1H, m), 6.30 (1H, d), 6.28 (1H, d), 6.26 (1H, s), 6.24 (1H, s), 5.89-5.82 (1H, m), 5.71 (1H, s), 5.57-5.53 (1H, m), 5.36 (1H, d), 5.04 (1H, d), 4.36 (1H, m), 3.44 (1H, m), remaining proton signals appear between 2.65-0.99 ppm; APCI-MS: m/z 559.2 [MH⁺].

### Example 12

### S-(Fluoromethyl)(4aS,4bR,5S,6aS,6bS,9aR,10aS,10bS,12S)-8-(1-benzofuran-2-yl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

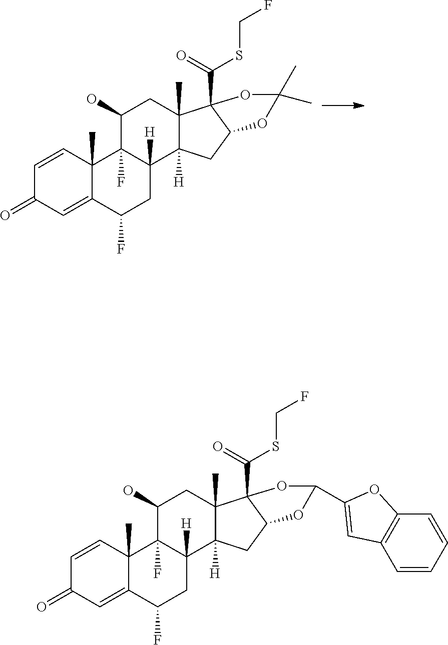

In a 4 mL vial intermediate 4 (50 mg, 0.1 mmol) was suspended in 2 mL of DCM. Perchloric acid (0.040 mL, 0.18 mmol) was added, the vial was capped and shaken for 5 min. 2-Formylbenzofuran (29.2 mg, 0.2 mmol) was added to the mixture and the vial was shaken for another 15 min. Triethylamine was added to neutralise the acid. The solvent was removed in vacuo and the crude product was purified by HPLC (MeCN-water 40-100% MeCN in 20 min) to afford the desired product (40 mg; 67%) as a colourless solid. The product was isolated as 2:1 mixture of 8-(R):8-(S) diastereomers according to ¹H-NMR spectroscopy.

¹H-NMR (400 MHz, CD₃CN) δ 7.66 (1H, d), 7.50 (1H, m), 7.39-7.17 (4H, m), 7.03 (1H, s), 6.83 (1H, m), 6.42 (1H, m), 6.31-6.25 (2H, m), 6.02 (1H, m), 5.89 (1H, m), 5.58-5.53 (1H, m), 5.47-5.43 (1H, m), 5.12 (1H, d), 4.34 (1H, m), 3.43 (1H, m), 2.75-2.61 (1H, m), 2.52-2.22 (2H, m), 1.89-1.66 (2H, m), 1.52 (3H, s), 1.04 (3H, s); APCI-MS: m/z 575.2 [MH⁺].

### Example 13

### S-(Fluoromethyl)(4aS,4bR,5S,6aS,6bS,9aR,10aS,10bS,12S)-4b,12-difluoro-5-hydroxy-4a,6a-dimethyl-8-(3-methyl-2-thienyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

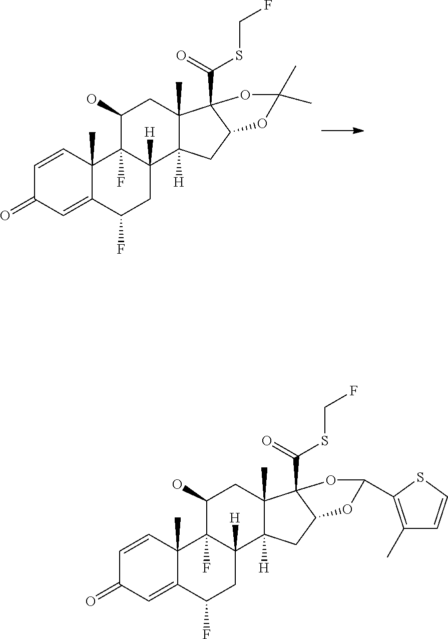

Same preparation following Example 12 using 3-methylthiophene-2-carbaldehyde. The product was isolated as a 10:1 mixture of 8-(R)- and 8-(S)-diastereomers according to ¹H-NMR spectroscopy.

¹H-NMR (400 MHz, CD₃CN) δ 7.36 (1H, d), 7.19 (1H, d), 6.86 (1H, d), 6.30-6.26 (1H, m), 6.24 (1H, s) 6.02-5.99 (1H, m), 5.90-5.85 (1H, d), 5.75-5.73 (1H, m), 5.58-5.54 (1H, m), 5.47-5.41 (1H, m), 5.02 (1H, s), 4.34-4.32 (1H, m), 3.43 (1H, m), 2.75-2.59 (1H, m), 2.75-2.60 (1H, m), 2.50-2.32 (1H, m), 2.25-2.08 (1H, m), 2.21 (3H, s), 1.82-1.61 (1H, m), 1.51 (3H, s), 1.02 (3H, s); APCI-MS: m/z 555.2 [MH⁺].

### Example 14

### S-(Fluoromethyl)(4aS,4bR,5S,6aS,6bS,9aR,10aS,10bS,12S)-4b,12-difluoro-5-hydroxy-4a,6a-dimethyl-2-oxo-8-[5-(trifluoromethyl)-2-furyl]-2,4a,4b,5,6,6a,9a,10,10a,10b,11,12-dodecahydro-6bH-naphtho[2′,1′:4,5]indeno[1,2-d][1,3]dioxole-6b-carbothioate

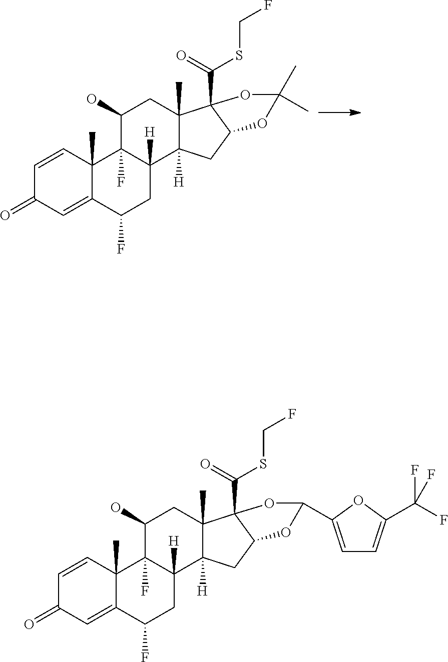

Same preparation following Example 12 using 5-(trifluoromethyl)furan-2-carbaldehyde. The product was isolated as a 1:8 mixture of 8-(R)- and 8-(S)-diastereomers according to ¹H-NMR spectroscopy.

¹H-NMR (400 MHz, CD₃CN) δ 7.20 (1H, d), 6.87 (1H, d), 6.53 (1H, m), 6.32 (1H, m), 6.24 (1H, s), 5.82-5.80 (1H, m), 5.69-5.67 (1H, m), 5.58-5.54 (1H, m), 5.56 (1H, m), 5.46-5.41 (1H, m), 5.36 (1H, d), 4.35-4.32 (1H, m), 3.42 (1H, m), 2.70-2.55 (1H, m), 2.38-2.30 (1H, m), 2.24-1.95 (2H, m), 1.77-1.64 (1H, m), 1.51 (3H, s), 0.99 (3H, s); APCI-MS: m/z 593.2 [MH⁺].

### Example 15

### S-(Fluoromethyl)(4aS,4bR,5S,6aS,6bS,9aR,10aS,10bS,12S)-4b,12-difluoro-5-hydroxy-4a,6a-dimethyl-2-oxo-8-(2-thienyl)-2,4a,4b,5,6,6a,9a,10,10a,10b,11,12-dodecahydro-6bH-naphtho[2′,1′:4,5]indeno[1,2-d][1,3]dioxole-6b-carbothioate

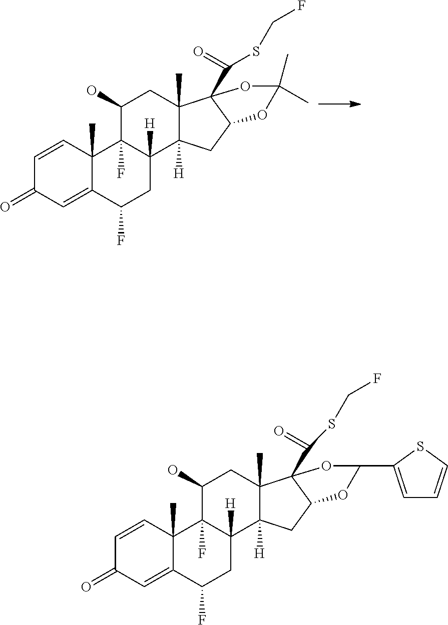

The same preparation was followed as in Example 1, using Intermediate 6. APCI-MS: m/z 541.2 [MH⁺].

### Example 16

### S-(Cyanomethyl)(4aS,4bR,5S,6aS,6bS,9aR,10aS,10bS,12S)-4b,12-difluoro-5-hydroxy-4a,6a-dimethyl-2-oxo-8-quinolin-6-yl-2,4a,4b,5,6,6a,9a,10,10a,10b,11,12-dodecahydro-6bH-naphtho[2′,1′:4,5]indeno[1,2-d][1,3]dioxole-6b-carbothioate

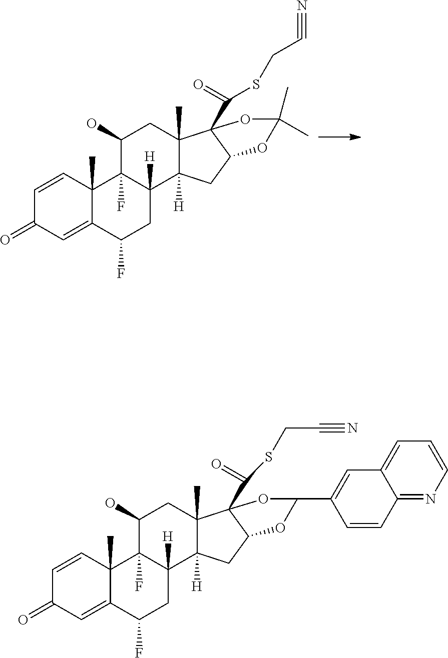

The same preparation was followed as in Example 1, using Intermediate 4.

¹H-NMR (400 MHz, CD₃CN) δ 8.92 (1H, m), 8.29 (1H, d), 8.13-7.94 (2H, m), 7.82 (1H, d), 7.74 (1H, m), 7.50 (1H, m), 7.24-7.16 (1H, m), 6.33-6.23 (2H, m), 5.82 (1H, s), 5.62-5.54 (1H, m), 5.51-5.43 (1H, m), 5.09 (1H, d), 4.38-4.32 (1H, m), 3.85-3.74 (2H, m), 2.79-2.55 (2H, m), 2.43-2.31 (2H, m), 1.96-1.69 (4H, m); 1.53 (3H, s), 1.22 (3H, s); APCI-MS: m/z 593.2 [MH⁺].

### Example 17

### S-(Fluoromethyl)(4aS,4bR,5S,6aS,6bS,9aR,10aS,10bS,12S)-4b,12-difluoro-5-hydroxy-4a,6a-dimethyl-8-(4-methylphenyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

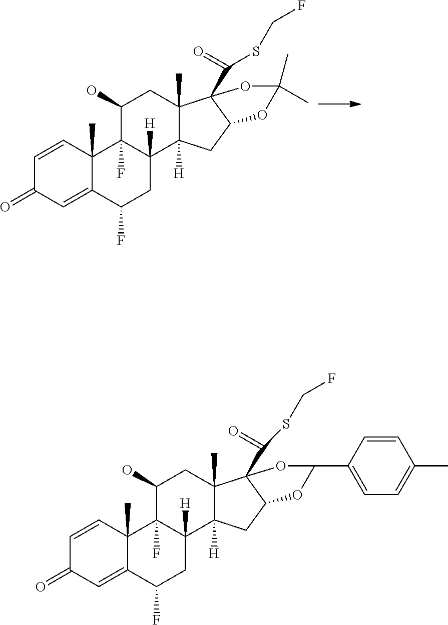

The same preparation was followed as in Example 1, using Intermediate 6.

¹H-NMR (400 MHz, CD₃CN) δ 7.36 (2H, d), 7.26-7.16 (3H, m), 6.30-6.24 (2H, m), 6.00 (1H, d), 5.87 (1H, d), 5.56 (1H, s), 5.01 (1H, d), 4.38-4.25 (1H, m), 3.55-3.39 (1H, m), 2.78-2.58 (2H, m), 2.34 (2H, d), 2.22-2.12 (2H, m), 1.85-1.61 (2H, m), 1.52 (3H, s), 1.28 (3H, s), 1.02 (3H, s); APCI-MS: m/z 549.2 [MH⁺].

### Example 18

### S-(Fluoromethyl)(4aS,4bR,5S,6aS,6bS,9aR,10aS,10bS,12S)-4b,12-difluoro-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate

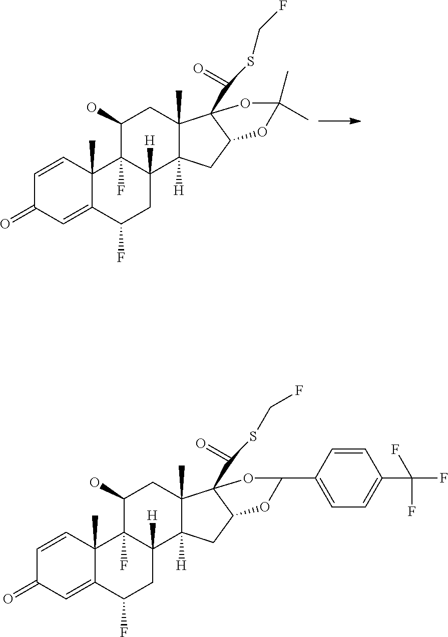

The same preparation was followed as in Example 1, using Intermediate 6. The compound was isolated as an 8:1 mixture of 8-(R)- and 8-(S)-diastereomers according to ¹H-NMR spectroscopy.

¹H-NMR (400 MHz, CD₃CN) δ 7.74 (2H, d), 7.66 (2H, d), 7.18 (1H, m), 6.32-6.22 (2H, m), 6.02 (1H, d), 5.89 (1H, m), 5.76 (1H, d), 5.68 (1H, s), 5.60-5.52 (1H, m), 5.48-5.40 (1H, m), 5.08 (1H, d), 4.33 (1H, d), 3.48 (1H, s), 2.77-2.58 (2H, m), 2.38-2.24 (2H, m), 2.16-2.07 (1H, m), 1.87-1.80 (2H, m), 1.75-1.60 (1H, m), 1.52 (3H, s), 1.04 (3H, s); APCI-MS: m/z 603.2 [MH⁺].

### Example 19

### S-(Cyanomethyl)(4aS,4bR,5S,6aS,6bS,9aR,10aS,10bS,12S)-4b,12-difluoro-5-hydroxy-4a,6a-dimethyl-2-oxo-8-[4-(trifluoromethoxy)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate

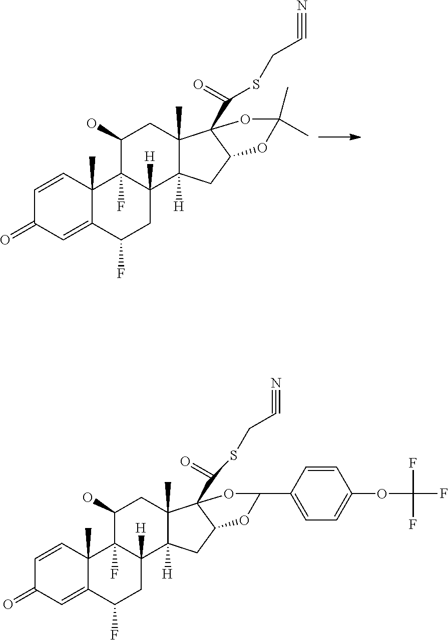

The same preparation was followed as in Example 1, using Intermediate 4. The compound was isolated as a 4:1 mixture of 8-(R)- and 8-(S)-diastereomers according to ¹H-NMR spectroscopy.

¹H-NMR (400 MHz, CD₃CN) δ 7.58 (2H, d), 7.34 (2H, d), 7.18 (1H, d), 6.30-6.26 (1H, m), 6.25 (1H, s), 5.66 (1H, s), 5.60-5.53 (1H, m), 5.48-5.41 (1H, m), 5.03 (1H, d), 4.36 (1H, s), 3.83-3.71 (2H, m), 2.78-2.56 (2H, m), 2.40-2.23 (2H, m), 2.10 (1H, s), 1.80 (1H, s), 1.52 (3H, s), 1.05 (3H, s); APCI-MS: m/z 626.2 [MH⁺].

### Example 20

### S-(Cyanomethyl)(4aS,4bR,5S,6aS,6bS,9aR,10aS,10bS,12S)-4b,12-difluoro-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate

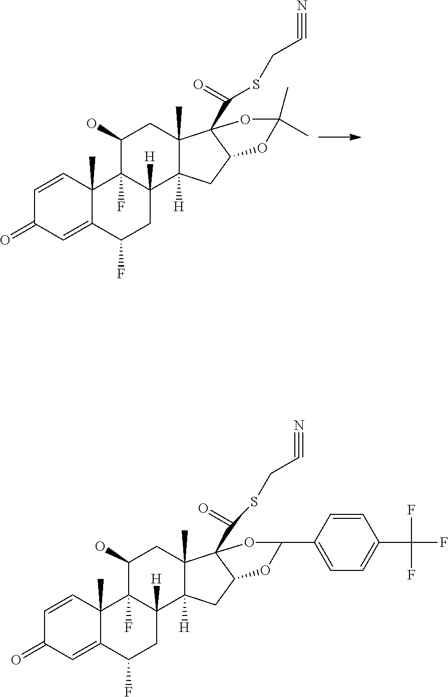

The same preparation was followed as in Example 1, using Intermediate 4. The compound was isolated as a 3:1 mixture of 8-(R)- and 8-(S)-diastereomers according to ¹H-NMR spectroscopy.

¹H-NMR (400 MHz, CD₃CN) δ 7.70 (2H, dd), 7.29-7.13 (2H, m), 6.34-6.21 (1H, m), 5.71 (1H, s), 5.63-5.50 (1H, m), 5.45 (1H, m), 5.05 (1H, d), 4.34 (1H, m), 3.78 (2H, m), 2.77-2.60 (2H, m), 2.40-2.23 (2H, m), 2.20-2.03 (1H, m), 1.91-1.57 (4H, m), 1.52 (3H, s), 1.13 (3H, s); APCI-MS: m/z 610.2 [MH⁺].

### Example 21

### S-(Fluoromethyl)(4aS,4bR,5S,6aS,6bS,9aR,10aS,10bS,12S)-4b,12-difluoro-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

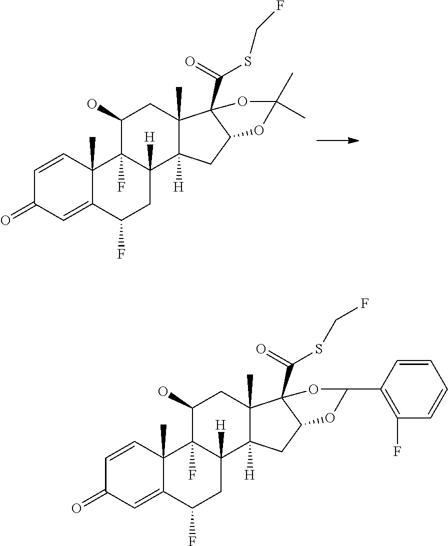

The same preparation was followed as in Example 1, using Intermediate 6. The compound was isolated as a 4:1 mixture of 8-(R)- and 8-(S)-diastereomers according to ¹H-NMR spectroscopy.

¹H-NMR (400 MHz, CD₃CN) δ 7.65-7.57 (1H, m), 7.51-7.43 (1H, m), 7.27-7.13 (2H, m), 6.28 (1H, d), 6.24 (1H, s), 6.02 (1H, d), 5.90-5.86 (1H, m), 5.88 (1H, d), 5.75-5.72 (1H, m), 5.60-5.53 (1H, m), 5.48-5.40 (1H, m), 5.05 (1H, d), 4.35-4.25 (1H, m), 3.65 (1H, m), the remaining proton signals appear between: 2.75-1.01 ppm; APCI-MS: m/z 553.2 [MH⁺].

### Example 22

### S-(Fluoromethyl)(4aS,4bR,5S,6aS,6bS,9aR,10aS,10bS,12S)-4b,12-difluoro-5-hydroxy-4a,6a-dimethyl-8-(2-methylphenyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

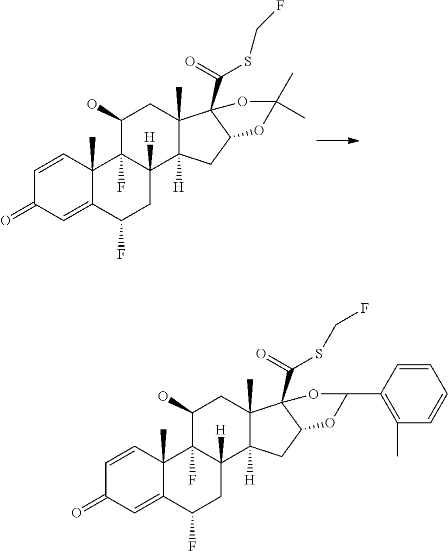

The same preparation was followed as in Example 1, using Intermediate 6. The compound was isolated as a 4:1 mixture of 8-(R)- and 8-(S)-diastereomers according to ¹H-NMR spectroscopy.

¹H-NMR (400 MHz, CD₃CN) δ 7.61 (1H, d), 7.33-7.12 (4H, m), 6.26 (2H, d), 6.23 (1H, s), 6.03 (1H, d), 5.89 (2H, t), 5.82 (1H, s), 5.75 (1H, d), 5.56 (1H, m), 5.46-5.41 (1H, m), 5.06 (1H, d), 4.31 (1H, dd), 3.45 (1H, s), 2.66 (2H, m), 2.37 (3H, s), 2.35-2.23 (2H, m), 1.63 (1H, m), 1.52 (3H, s), 1.03 (3H, s); APCI-MS: m/z 549.2 [MH⁺].

### Example 23

### S-(Cyanomethyl)(4aS,4bR,5S,6aS,6bS,9aR,10aS,10bS,12S)-4b,12-difluoro-8-(3-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

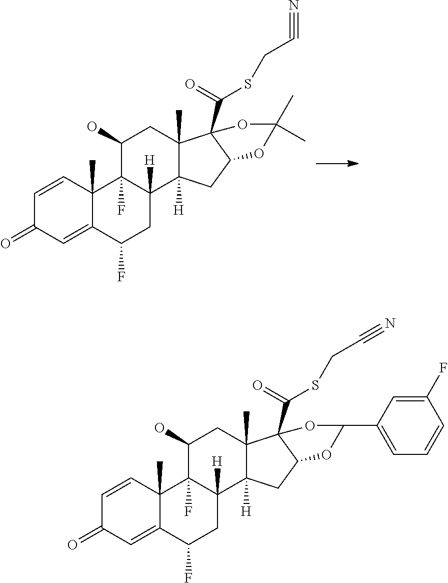

The same preparation was followed as in Example 1, using Intermediate 4. The compound was isolated as 8:1 mixture of 8-(R)- and 8-(S)-diastereomers according to ¹H-NMR spectroscopy.

¹H-NMR (400 MHz, CD₃CN) δ 7.49-7.40 (1H, m), 7.34-7.29 (1H, m), 7.25-7.16 (2H, m), 6.29 (1H, d), 6.24 (1H, s), 5.64 (1H, s), 5.60-5.53 (1H, m), 5.48-5.40 (1H, m), 5.03 (1H, d), 4.37-4.31 (1H, m), 3.77 (2H, m), 3.48 (1H, m), 2.76-2.60 (2H, m), 2.38-2.24 (2H, m), 2.12 (1H, d), 1.86-1.62 (4H, m), 1.52 (3H, s), 1.05 (3H, s); APCI-MS: m/z 560.6 [MH⁺].

### Example 24

### S-(Cyanomethyl)(4aS,4bR,5S,6aS,6bS,9aR,10aS,10bS,12S)-4b,12-difluoro-5-hydroxy-4a,6a-dimethyl-8-(4-methylphenyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

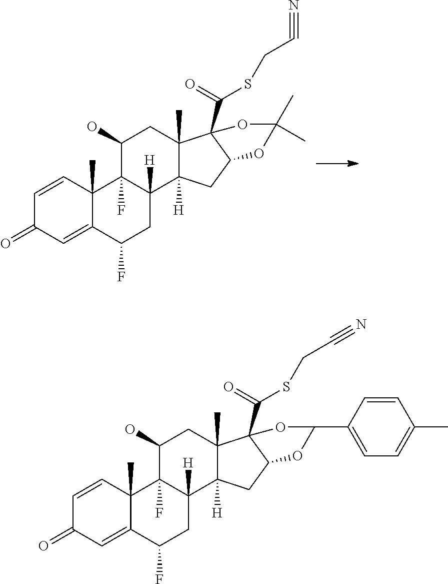

The same preparation was followed as in Example 1, using Intermediate 4. The compound was isolated as 8:1 mixture of 8-(R)- and 8-(S)-diastereomers according to ¹H-NMR spectroscopy.

¹H-NMR (400 MHz, CD₃CN) δ 7.19 (1H, d), 6.28 (1H, d), 6.23 (1H, s), 5.55 (1H, m), 5.47 (1H, s), 5.47-5.40 (1H, m), 5.00 (1H, d), 4.38-4.29 (1H, m), 3.74 (2H, m), 3.45 (1H, t), 2.62 (2H, m), 2.32 (2H, m), 2.20-2.02 (4H, m), 1.79-1.59 (4H, m), 1.51 (3H, s), 1.22 (3H, s), 0.96 (3H, s); APCI-MS: m/z 556.2 [MH⁺].

### Example 25

### S-(Cyanomethyl)(4aS,4bR,5S,6aS,6bS,9aR,10aS,10bS,12S)-8-(3-cyanophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

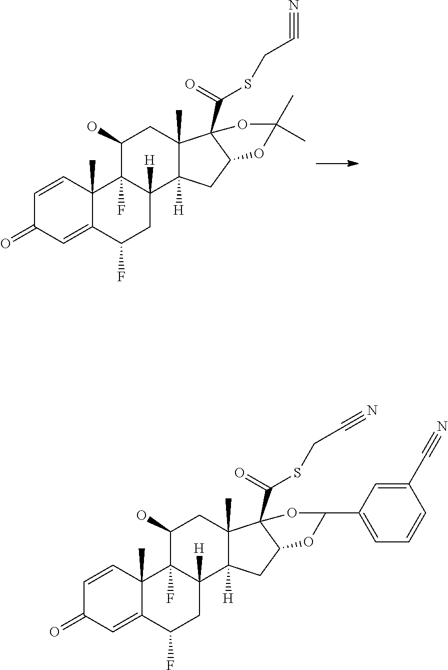

The same preparation was followed as in Example 1, using Intermediate 4. The compound was isolated as 3:1 mixture of 8-(R)- and 8-(S)-diastereomers according to ¹H-NMR spectroscopy.

¹H-NMR (400 MHz, CD₃CN) δ 7.83-7.74 (2H, m), 7.61 (1H, d), 7.18 (1H, m), 6.30-6.24 (2H, m), 5.68 (1H, s), 5.62-5.51 (1H, m), 5.50-5.39 (1H, m), 5.06 (1H, d), 4.35 (1H, d), 3.78 (2H, m), 2.73-2.62 (2H, m), 2.35-2.24 (2H, m), 1.91-1.59 (3H, m), 1.52 (3H, s), 1.06 (3H, s); APCI-MS: m/z 567.2 [MH⁺].

### Example 26

### S-(Cyanomethyl)(4aS,4bR,5S,6aS,6bS,9aR,10aS,10bS,12S)-4b,12-difluoro-8-(4-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

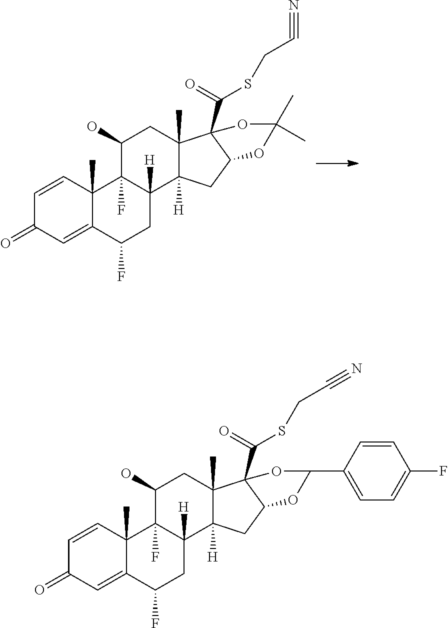

The same preparation was followed as in Example 1, using Intermediate 4. The compound was isolated as 8:1 mixture of 8-(R)- and 8-(S)-diastereomers after purification according to ¹H-NMR spectroscopy.

¹H-NMR (400 MHz, CD₃CN) δ 7.54-7.49 (2H, m), 7.23-7.12 (3H, m), 6.30-6.23 (2H, m), 5.60 (1H, s), 5.59-5.54 (1H, m), 5.45 (1H, m), 5.01 (1H, d), 4.36-4.31 (1H, m), 3.77 (2H, m), 3.49 (1H, s), 2.78-2.58 (2H, m), 2.39-2.27 (2H, m), 1.86-1.52 (4H, m), 1.45 (3H, s), 1.05 (3H, s); APCI-MS: m/z 560.2 [MH⁺].

### Example 27

### S-(Cyanomethyl)(4aS,4bR,5S,6aS,6bS,9aR,10aS,10bS,12S)-4b,12-difluoro-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

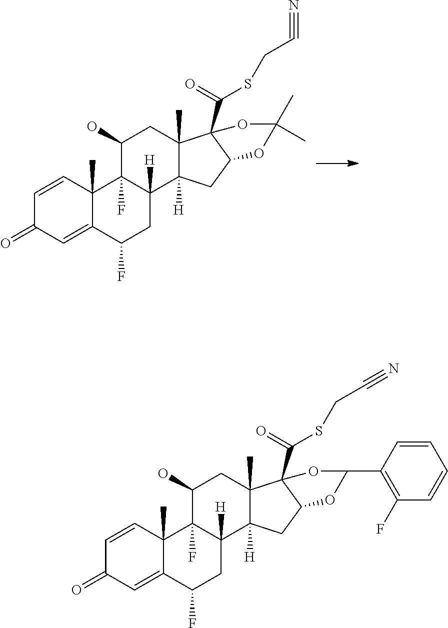

The same preparation was followed as in Example 1, using Intermediate 4. APCI-MS: m/z 560.6 [MH⁺].

### Example 28

### S-(Cyanomethyl)(4aS,4bR,5S,6aS,6bS,9aR,10aS,10bS,12S)-8-(4-chlorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′,4,5]indeno[1,2-d][1,3]dioxole-6b-carbothioate

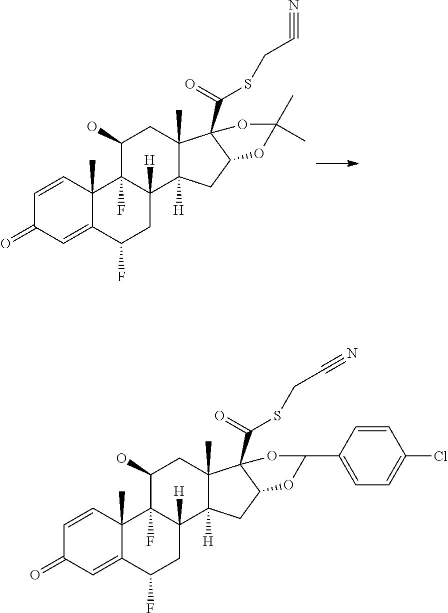

The same preparation was followed as in Example 1, using Intermediate 4. The compound was isolated as 8:1 mixture of 8-(R)- and 8-(S)-diastereomers according to ¹H-NMR spectroscopy.

¹H-NMR (400 MHz, CD₃CN) δ 7.48-7.39 (4H, m), 7.18 (1H, d), 6.31-6.20 (2H, m), 5.62 (1H, s), 5.59-5.53 (1H, m), 5.48-5.42 (1H, m), 5.01 (1H, d), 4.35 (1H, m), 3.77 (2H, q), 3.48 (1H, m), 2.77-2.59 (2H, m), 2.39-2.22 (2H, m), 1.86-1.76 (2H, m), 1.68 (2H, m), 1.51 (3H, s), 1.04 (3H, s); APCI-MS: m/z 575.1 [MH⁺].

### Example 29

### S-(Fluoromethyl)(4aS,4bR,5S,6aS,6bS,9aR,10aS,10bS,12S)-8-(4-chlorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

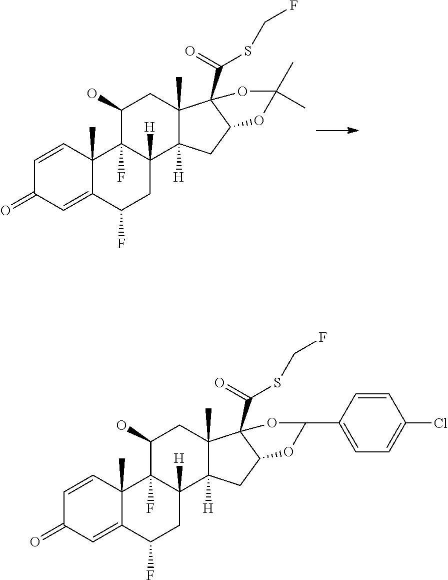

The same preparation was followed as in Example 1, using Intermediate 6. The compound was isolated as 4:1 mixture of 8-(R)- and 8-(S)-diastereomers according to ¹H-NMR spectroscopy.

¹H-NMR (400 MHz, CD₃CN) δ 7.45 (4H, m), 7.17 (1H, d), 6.25 (2H, m), 6.01 (1H, d), 5.92-5.85 (1H, m), 5.75 (1H, d), 5.59 (1H, s), 5.59-5.52 (1H, m), 5.48-5.41 (1H, m), 5.04 (1H, d), 4.36-4.29 (1H, m), 3.46-3.39 (1H, m), 2.78-2.58 (2H, m), 2.44-2.24 (2H, m), 1.86 (2H, m), 1.73-1.62 (1H, m), 1.52 (3H, s), 1.02 (3H, s); APCI-MS: m/z 570.0 [MH⁺].

### Example 30

### S-(Fluoromethyl)(4aS,4bR,5S,6aS,6bS,9aR,10aS,10bS,12S)-4b,12-difluoro-5-hydroxy-4a,6a-dimethyl-2-oxo-8-phenyl-2,4a,4b,5,6,6a,9a,10,10a,10b,11,12-dodecahydro-6bH-naphtho[2′,1′:4,5]indeno[1,2-d][1,3]dioxole-6b-carbothioate

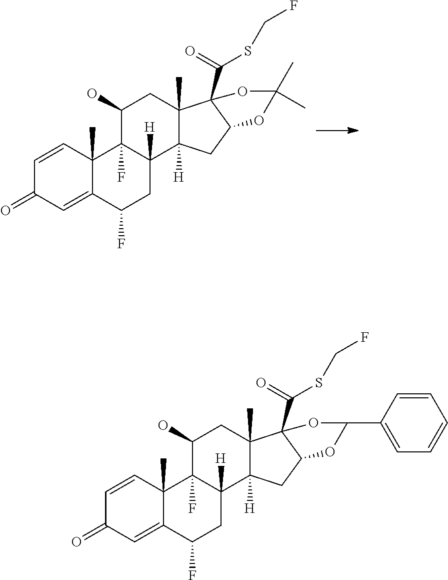

The same preparation was followed as in Example 1, using Intermediate 6. The compound was isolated as 3:1 mixture of 8-(R)- and 8-(S)-diastereomers diastereomers according to ¹H-NMR spectroscopy.

¹H-NMR (400 MHz, CD₃CN) δ 7.51-7.40 (5H, m), 7.18 (1H, d), 6.34-6.18 (2H, m), 6.01 (1H, d), 5.91-5.84 (1H, m), 5.80 (1H, d), 5.60 (1H, s), 5.59-5.52 (1H, m), 5.49-5.42 (1H, m), 5.04 (1H, d), 4.36-4.29 (1H, m), 4.15-4.08 (1H, m), remaining proton signals appear between 2.72-1.03 ppm; APCI-MS: m/z 535.5 [MH⁺].

### Example 31

### S-(Cyanomethyl)(4aS,4bR,5S,6aS,6bS,9aR,10aS,10bS,12S)-4b,12-difluoro-5-hydroxy-4a,6a-dimethyl-2-oxo-8-phenyl-2,4a,4b,5,6,6a,9a,10,10a,10b,11,12-dodecahydro-6bH-naphtho[2′,1′:4,5]indeno[1,2-d][1,3]dioxole-6b-carbothioate

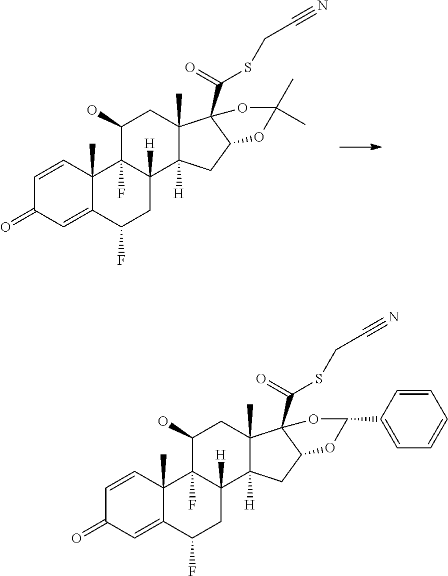

The same preparation was followed as in Example 1, using Intermediate 4.

¹H-NMR (400 MHz, CD₃CN) δ 7.49-7.42 (5H, m), 7.21-7.14 (1H, m), 6.25 (1H, s), 5.63 (1H, s), 5.60-5.53 (1H, m), 5.48-5.41 (1H, m), 5.01 (1H, d), 4.38-4.31 (1H, m), 3.77 (2H, q) 2.79-2.58 (1H, m), 2.40-2.30 (1H, m), 2.10 (3H, s), 1.84-1.58 (2H, m), 1.52 (3H, s), 1.05 (3H, s); APCI-MS: m/z 542.6 [MH⁺].

### Example 32

### S-(Fluoromethyl)(4aS,4bR,5S,6aS,6bS,9aR,10aS,10bS,12S)-8-(2,3-dihydro-1-benzofuran-7-yl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

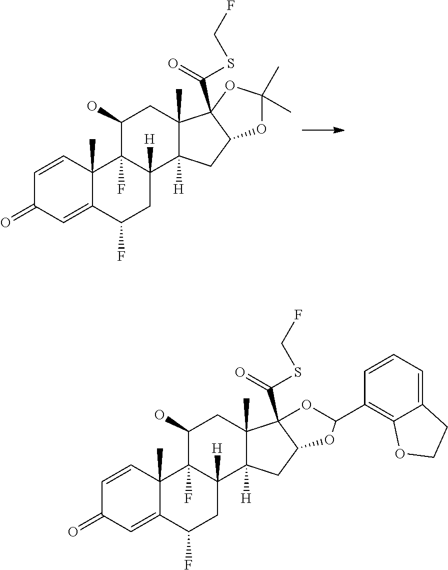

To a suspension of Intermediate 6 (50 mg, 0.1 mmol) in dichloromethane (2 ml) was added perchloric acid (70% wt., 30 μl). The mixture was vigorously shaken for 2 min., then 2,3-dihydrobenzofuran-7-carbaldehyde (76 mg, 0.5 mmol) was added. The mixture was briefly shaken and then stirred at RT for 3 hrs. Triethylamine (100 μl) was added, and the mixture was concentrated in vacuo. The crude material was purified on HPLC (acetonitrile/water, gradient of 50% to 90%) to give 17 mg (29 μmol, 29%) of the target compound as a 88:12 mixture of the 8-(R)- and 8-(S)-diastereomers, according to ¹H-NMR spectroscopy.

¹H-NMR (400 MHz, CD₃CN) δ 7.28-7.16 (m), 7.07 (1H, d, S), 6.85 (1H, t, R), 6.79 (1H, t, S), 6.32-6.23 (m), 6.01 (d), 5.87 (t), 5.73 (m), 5.58 (m), 5.45 (m), 5.40 (1H, d, S), 4.99 (1H, d, R), 4.71 (m, 4.52 (m), 4.33 (m), 3.43 (m), 3.17 (m), 2.75-2.30 (m), 2.15 (m, partially covered with signal of H₂O), 1.88-1.60 (m), 1.52 (s), 1.02 (s); APCI-MS: m/z 577 [MH⁺].

### Example 33

### S-(Fluoromethyl)(4aS,4bR,5S,6aS,6bS,9aR,10aS,10bS,12S)-8-(3,5-dimethylisoxazol-4-yl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

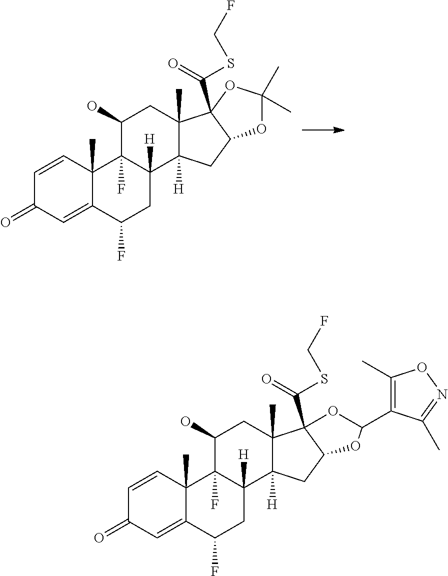

Prepared from intermediate 6 and 3,5-dimethylisoxazole-4-carbaldehyde, following the procedure described for Example 32. The compound was isolated as a mixture of 8-(R)- and 8-(S)-diastereomers in a ratio of 82:18, according to ¹H-NMR spectroscopy.

¹H-NMR (400 MHz, CD₃CN) δ 7.19 (dd), 6.32-6.18 (m), 6.01 (d), 5.91-5.64 (m), 5.60-5.41 (m), 5.39 (1H, d, S), 4.98 (1H, d, R), 2.40-2.07 (m), 1.90-1.54 (m), 1.52 (s), 1.01 (s); APCI-MS: m/z 554 [MH⁺].

### Example 34

### S-(Fluoromethyl)(4aR,4bS,5S,6aS,6bS,9aR,10aS,10bS)-8-furan-2-yl-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

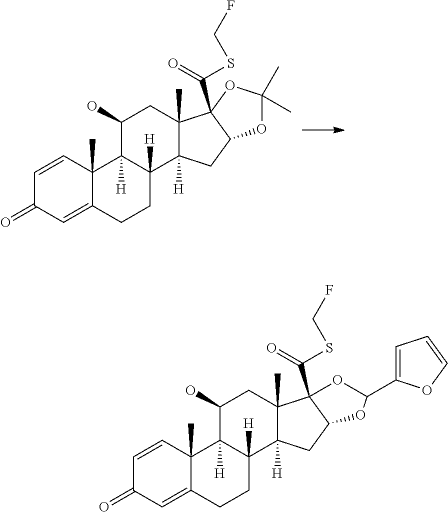

Prepared from intermediate 18 and furan-2-carbaldehyde, following the procedure described for Example 32. The compound was isolated as a mixture of 8-(R)- and 8-(S)-diastereomers in a ratio of 77:23, according to ¹H-NMR spectroscopy.

¹H-NMR (400 MHz, CD₃CN) δ 7.53 (1H, d, R), 7.43 (1H, d, S), 7.28 (m), 6.61 (1H, d, R), 6.44 (1H, dd, R), 6.41 (1H, d, S), 6.34 (1H, dd, S), 6.18 (m), 5.97-5.58 (m), 5.31 (d1H, d, S), 4.98 (1H, d, R), 4.43 (m), 2.84 (m), 2.60 (m), 2.37-2.05 (m), 1.90-1.68 (m), 1.44 (3H, s, R), 1.43 (3H, s, S), 1.20-1.07 (m), 1.02 (3H, s, R), 1.00 (3H, s, S); APCI-MS: m/z 489 [MH⁺].

### Example 35

### S-(Fluoromethyl)(4aR,4bS,5S,6aS,6bS,8R,9aR,10aS,10bS)-5-hydroxy-4a,6a-dimethyl-2-oxo-8-thiophen-2-yl-2,4a,4b,5,6,6a,9a,10,10a,10,11,12-dodecahydro-6bH-naphtho[2′,1′:4,5]indeno[1,2-d][1,3]dioxole-6b-carbothioate

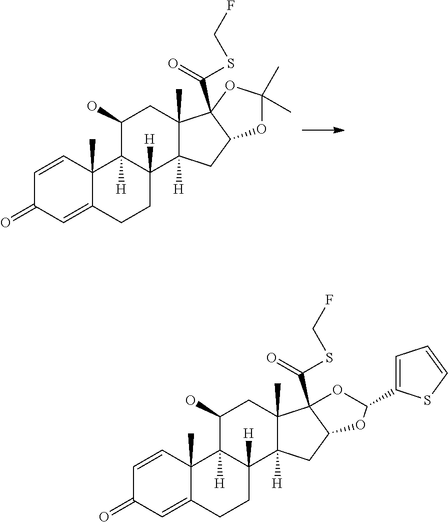

Prepared from intermediate 18 and 3,5-dimethylisoxazole-4-carbaldehyde, following the procedure described for Example 32. The compound was isolated as the 8-(R)-diastereomer (>99%), according to ¹H-NMR spectroscopy.

¹H-NMR (400 MHz, CD₃CN) δ 7.49 (1H, d) 7.28 (1H, d), 7.23 (1H, dd), 7.02 (1H, dd), 6.17 (1H, dd), 6.01-5.91 (3H, m), 5.86 (1H, d), 5.74 (1H, d), 4.98 (1H, d), 4.43 (1H, m), 2.87 (1H, d), 2.61 (1H, m), 2.38-2.08 (3H, m), 1.87-1.74 (5H, m), 1.44 (3H, s), 1.19-1.07 (2H, m), 1.02 (3H, s); APCI-MS: m/z 505 [MH⁺].

### Example 36

### S-(Cyanomethyl)(4aR,4bS,5S,6aS,6bS,9aR,10aS,10bS)-8-(2,4-dimethylphenyl)-5-hydroxy-4a,6a-dimethyl-2-oxo-2,4a,4b,5,6,6a,9a,10,10a,10,11,12-dodecahydro-6bH-naphtho[2′,1′:4,5]indeno[1,2-d][1,3]dioxole-6b-carbothioate

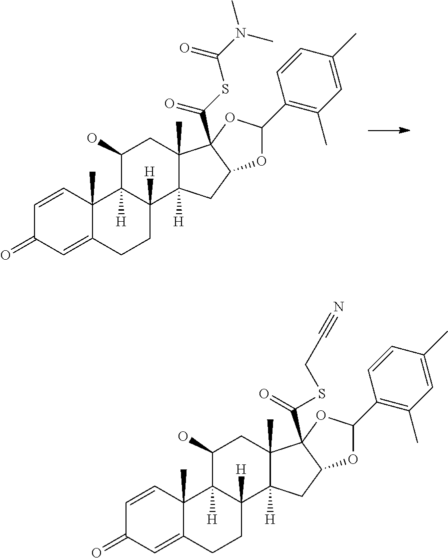

To a stirred solution of Intermediate 12 (88 mg, 0.16 mmol) in N,N-dimethylacetamide (2 ml) was added sodium hydrogensulfide (87 mg, 1.6 mmol) and stirring was continued for 1 hour at RT. Then the mixture was poured into aq. HCl (1M, 10 ml) and extracted with ethyl acetate (3×15 ml). The combined organic extracts were washed with water (20 ml), and dried with Na₂SO₄. After filtration the solution was evaporated to dryness to afford (4aR,4bS,5S,6aS,6bS,9aR,10aS,10bS)-8-(2,4-dimethylphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioic S-acid (60 mg, 0.12 mmol), which was dissolved in dichloromethane (2 mL). Triethylamine (0.1 ml) was added, followed by 2-bromoacetonitrile (43.6 mg, 0.36 mmol). The mixture was stirred at RT for 1 hour, and concentrated in vacuo. The crude material was purified on HPLC (acetonitrile/water, gradient of 50% to 90%) to give 22 mg (41 μmol, 26%) of the target compound as a 84:16 mixture of the 8-(R)- and 8-(S)-diastereomers, according to ¹H-NMR spectroscopy.

¹H-NMR (400 MHz, CD₃CN) δ 7.48 (1H, d, R), 7.28 (m), 7.03 (m), 6.97 (1H, d, S), 6.33 (1H, d, S), 6.16 (m), 5.94 (m), 5.78 (s), 5.36 (1H, d, S), 4.97 (1H, d, R), 4.44 (1H, m, S), 4.40 (1H, m, R), 3.75 (2H, dd, R), 3.57 (2H, dd, S), 2.89 (m), 2.69 (s), 2.59 (m), 2.38-2.17 (m), 2.14 (s), 2.09 (m), 1.91-1.64 (m), 1.44 (3H, s, S), 1.43 (3H, s, R), 1.17-1.02 (m); APCI-MS: m/z 534 [MH⁺].

### Example 37

### S-(Cyanomethyl)(4aR,4bS,5S,6aS,6bS,9aR,10aS,10bS)-8-(2-fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

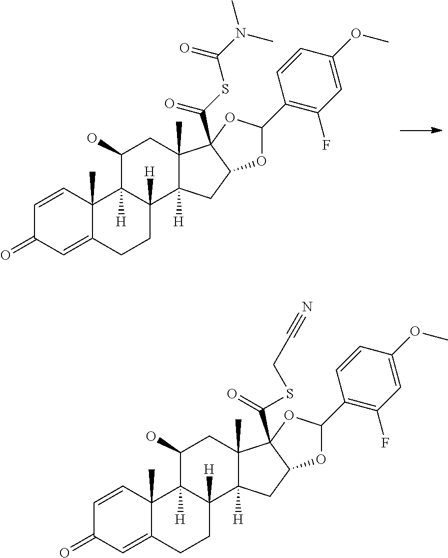

Prepared from Intermediate 11 and bromoacetonitrile, following the procedure described for Example 36. The compound was isolated as a mixture of 8-(R)- and 8-(S)-diastereomers in a ratio of 85:15, according to ¹H-NMR spectroscopy.

¹H-NMR (400 MHz, CD₃CN) δ 7.52 (1H, t, R), 7.29 (m), 6.79 (1H, dd, R), 6.69 (m), 6.38 (1H, s, S), 6.17 (m), 5.94 (br. s), 5.80 (1H, s, R), 5.36 (1H, d, S), 4.95 (1H, d, R), 4.42 (br.s), 3.80 (3H, s, R), 3.78 (3H, s, S), 3.74 (2H, dd, R), 3.60 (2H, dd, S), 2.91 (br. s), 2.60 (m), 2.37-2.05 (m), 1.92-1.71 (m), 1.44 (s), 1.22-1.08 (m), 1.04 (m); APCI-MS: m/z 554 [MH⁺].

### Example 38

### S-Methyl (4aR,4bS,5S,6aS,6bS,8R,9aR,10aS,10bS)-8-(2-fluoro-4-methoxyphenyl)-5-hydroxy-4a,6a-dimethyl-2-oxo-2,4a,4b,5,6,6a,9a,10,10a,10,11,12-dodecahydro-6bH-naphtho[2′,1′:4,5]indeno[1,2-d][1,3]dioxole-6b-carbothioate and S-Methyl (4aR,4bS,5S,6aS,6bS,8S,9aR,10aS,10bS)-8-(2-fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

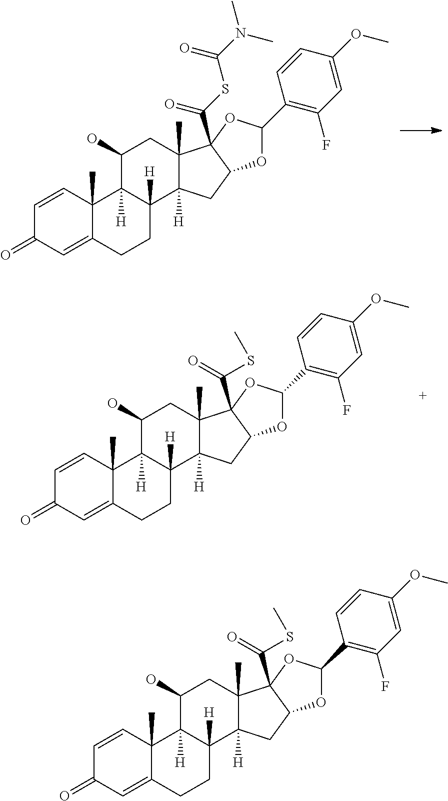

Prepared from Intermediate 11 and iodomethane, following the procedure described for Example 36. The obtained 8-(R)- and 8-(S)-diastereomers, obtained in a ratio of 4:1, were separated by HPLC yielding S-Methyl (4aR,4bS,5S,6aS,6bS,8R,9aR,10aS,10bS)-8-(2-fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate:

¹H-NMR (400 MHz, CD₃CN) δ 7.52 (1H, t), 7.29 (1H, d), 6.78 (1H, dd), 6.70 (1H, dd), 6.17 (1H, dd), 5.94 (1H, s), 5.75 (1H, s, 4.94 (1H, d), 4.42 (1H, m), 3.80 (3H, s), 2.82 (1H, br.s), 2.60 (1H, m), 2.33 (1H, m), 2.30 (3H, s), 2.27-2.06 (3H, m), 1.86-1.71 (5H, m), 1.44 (3H, s), 1.14 (2H, dd), 0.99 (3H, s); APCI-MS: m/z 529 [MH⁺].

As the second peak was isolated:

### S-Methyl (4aR,4bS,5S,6aS,6bS,8S,9aR,10aS,10bS)-8-(2-fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

¹H-NMR (400 MHz, CD₃CN) δ 7.31 (2H, m), 6.67 (2H, m), 6.34 (1H, s), 6.17 (1H, dd), 5.94 (1H, s), 5.35 (1H, d), 4.43 (1H, m), 3.78 (3H, s), 3.59 (1H, br.s), 2.80 (1H, d), 2.60 (1H, m), 2.34 (1H, m), 2.24-2.06 (5H, m), 1.92-1.69 (4H, m), 1.45-1.39 (4H, m), 1.30-1.12 (4H, m), 0.98 (3H, s); APCI-MS: m/z 529 [MH⁺].

### Example 39

### S-(Cyanomethyl)(4aR,4bS,5S,6aS,6bS,9aR,10aS,10bS)-5-hydroxy-4a,6a-dimethyl-2-oxo-8-[4-(trifluoromethyl)phenyl]-2,4a,4b,5,6,6a,9a,10,10a,10,11,12-dodecahydro-6bH-naphtho[2′,1′:4,5]indeno[1,2-d][1,3]dioxole-6b-carbothioate

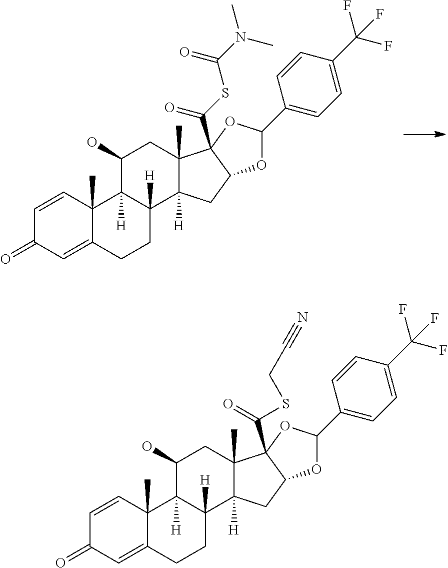

Prepared from Intermediate 13 and bromoacetonitrile, following the procedure described for Example 36. The compound was isolated as a mixture of 8-(R)- and 8-(S)-diastereomers in a ratio of 1:1, according to ¹H-NMR spectroscopy.

¹H-NMR (400 MHz, CD₃CN) δ 7.76-7.59 (m), 7.29 (dd), 6.23 (s), 6.18 (ddd), 5.95 (d), 5.67 (s), 5.39 (1H, d, S), 5.01 (1H, d, R), 4.43 (m), 3.76 (dd), 3.64 (dd), 2.91 (m), 2.61 (m), 2.39-1.69 (m), 1.45 (s), 1.44 (s), 1.28-1.09 (m), 1.08 (s), 1.05 (s); APCI-MS: m/z 574 [MH⁺].

### Example 40

### S-(Fluoromethyl)(4aR,4bS,5S,6aS,6bS,9aR,10aS,10bS)-5-hydroxy-4a,6a-dimethyl-2-oxo-8-[4-(trifluoromethyl)phenyl]-2,4a,4b,5,6,6a,9a,10,10a,10,11,12-dodecahydro-6bH-naphtho[2′,1′:4,5]indeno[1,2-d][1,3]dioxole-6b-carbothioate

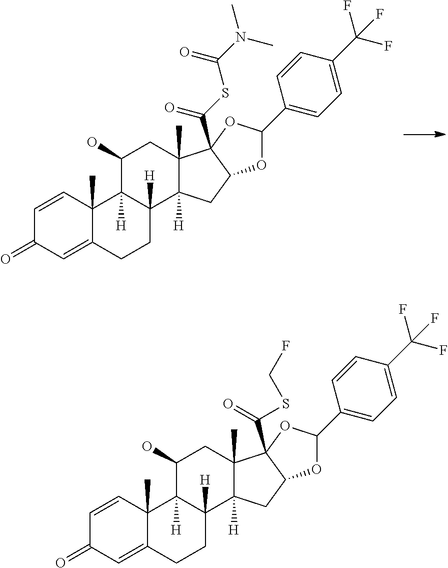

Prepared from Intermediate 13 and bromofluoromethane, following the procedure described for Example 36. The compound was isolated as a mixture of 8-(R)- and 8-(S)-diastereomers in a ratio of 1:1, according to ¹H-NMR spectroscopy.

¹H-NMR (400 MHz, CD₃CN) δ 7.75-7.65 (m), 7.59 (d), 7.29 (dd), 6.23 (s), 6.18 (ddd), 6.02-5.85 (m), 5.75 (dt), 5.62 (m), 5.40 (1H, d, S), 5.03 (1H, d, R), 4.43 (m), 2.87 (m), 2.61 (m), 2.39-1.72 (m), 1.44 (s), 1.44 (s), 1.28-1.09 (m), 1.05 (s), 1.03 (s); APCI-MS: m/z 567 [MH⁺].

### Example 41

### S-Methyl (4aR,4bS,5S,6aS,6bS,9aR,10aS,10bS)-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate

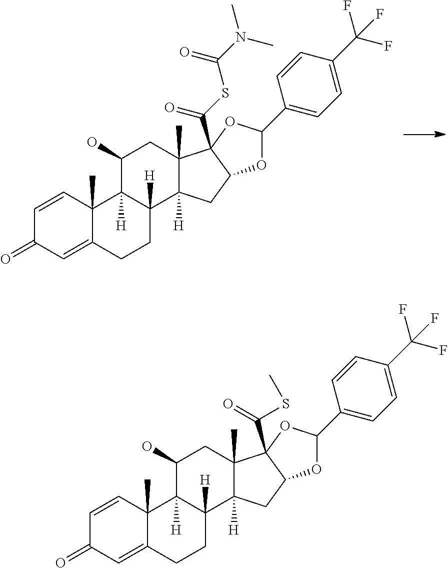

Prepared from Intermediate 13 and iodomethane, following the procedure described for Example 36. The compound was isolated as a mixture of 8-(R)- and 8-(S)-diastereomers in a ratio of 53:47, according to ¹H-NMR spectroscopy.

¹H-NMR (400 MHz, CD₃CN) δ 7.69 (m), 7.61 (d), 7.29 (dd), 6.18 (m), 5.94 (m), 5.62 (s), 5.37 (1H, d, S), 5.01 (1H, d, R), 4.43 (m), 2.81 (m), 2.60 (m), 2.34 (m), 2.32 (s), 2.26-2.04 (m), 1.92-1.72 (m), 1.45 (s), 1.44 (s), 1.27-1.09 (m), 1.02 (s), 1.00 (s); APCI-MS: m/z 549 [MH⁺].

### Example 42

### S-(Cyanomethyl)(4aR,4bS,5S,6aS,6bS,9aR,10aS,10bS)-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

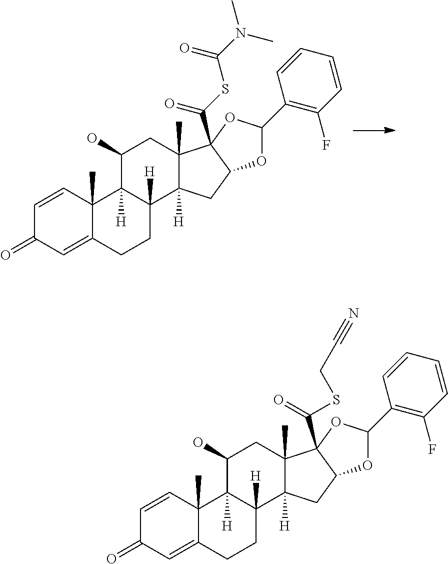

Prepared from Intermediate 14 and bromoacetonitrile, following the procedure described for Example 36. The compound was isolated as a mixture of 8-(R)- and 8-(S)-diastereomers in a ratio of 67:33, according to ¹H-NMR spectroscopy.

¹H-NMR (400 MHz, CD₃CN) δ 7.65 (1H, td, R), 7.50-7.37 (m), 7.31-7.22 (m), 7.19-7.07 (m), 6.45 (1H, s, S), 6.18 (1H, dd, S), 6.17 (1H, dd, R), 5.94 (m), 5.88 (1H, s, R), 5.40 (1H, d, S), 4.99 (1H, d, R), 4.43 (m), 3.75 (2H, dd, R), 3.58 (2H, dd, S), 2.90 (m), 2.60 (td), 2.38-2.05 (m), 1.92-1.70 (m), 1.44 (s), 1.24-1.08 (m), 1.05 (3H, s, R), 1.04 (3H, s, S); APCI-MS: m/z 524 [MH⁺].

### Example 43

### S-(Fluoromethyl)(4aR,4bS,5S,6aS,6bS,9aR,10aS,10bS)-8-(2-fluorophenyl)-5-hydroxy-4a,6a-dimethyl-2-oxo-2,4a,4b,5,6,6a,9a,10,10a,10,11,12-dodecahydro-6bH-naphtho[2′,1′:4,5]indeno[1,2-d][1,3]dioxole-6b-carbothioate

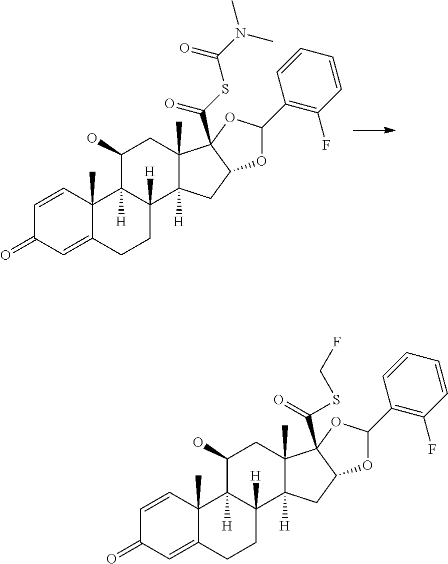

Prepared from Intermediate 14 and bromofluoromethane, following the procedure described for Example 36. The compound was isolated as a mixture of 8-(R)- and 8-(S)-diastereomers in a ratio of 66:34, according to ¹H-NMR spectroscopy.

¹H-NMR (400 MHz, CD₃CN) δ 7.65 (1H, td, R), 7.50-7.36 (m), 7.32-7.22 (m), 7.18-7.05 (m), 6.44 (1H, s, S), 6.17 (m), 6.03-5.56 (m), 5.40 (1H, d, S), 5.01 (1H, d, RS), 4.43 (m), 2.85 (s), 2.60 (td), 2.39-2.05 (m), 1.92-1.71 (m), 1.44 (s), 1.21-1.11 (m), 1.03 (3H, s, R), 1.02 (3H, s, S); APCI-MS: m/z 517 [MH⁺].

### Example 44

### S-Methyl (4aR,4bS,5S,6aS,6bS,9aR,10aS,10bS)-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate

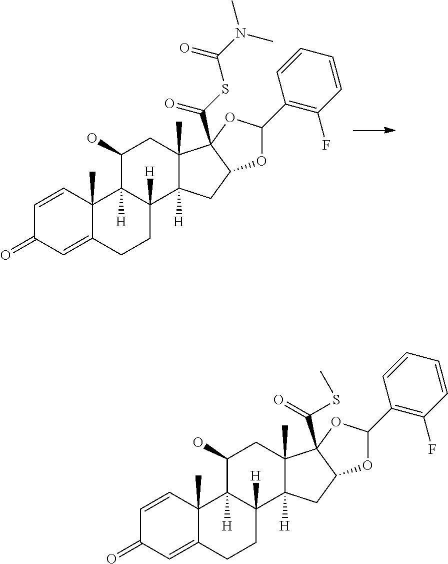

Prepared from Intermediate 14 and iodomethane, following the procedure described for Example 36. The compound was isolated as a mixture of 8-(R)- and 8-(S)-diastereomers in a ratio of 68:32, according to ¹H-NMR spectroscopy.

¹H-NMR (400 MHz, CD₃CN) δ 7.65 (1H, td, R), 7.49-7.36 (m), 7.31-7.21 (m), 7.18-7.05 (m), 6.42 (1H, s, S), 6.17 (m), 5.94 (m), 5.84 (1H, s, R), 5.38 (1H, d, S), 4.99 (1H, d, R), 4.42 (m), 2.80 (m), 2.60 (td), 2.38-2.04 (m), 1.92-1.70 (m), 1.44 (s), 1.28-1.07 (m), 1.00 (3H, s, R), 0.99 (3H, s, S); APCI-MS: m/z 499 [MH⁺].

### Human Glucocorticoid Receptor (GR) Assay

The assay is based on a commercial kit from Panvera/Invitrogen (Part number P2893). The assay technology is fluorescence polarization. The kit utilises recombinant human GR (Panvera, Part number P2812), a Fluoromone™ labelled tracer (GS Red, Panvera, Part number P2894) and a Stabilizing Peptide 10× (Panvera, Part number P2815). The GR and Stabilizing Peptide reagents are stored at −70° C. while the GS Red is stored at −20° C. Also included in the kit are 1M DTT (Panvera, Part number P2325, stored at −20° C.) and GR Screening buffer 10× (Panvera, Part number P2814, stored at −70° C. initially but once thawed stored at room temperature). Avoid repeated freeze/thaws for all reagents. The GR Screening buffer 10× comprises 100 mM potassium phosphate, 200 mM sodium molybdate, 1 mM EDTA and 20% DMSO.

Test compounds (1 μL) and controls (1 μL) in 100% DMSO were added to black polystyrene 384-well plates (Greiner low volume black flat-bottom, part number 784076). 0% control was 100% DMSO and 100% control was 10 μM Dexamethasone. Background solution (8 μL; assay buffer 10×, Stabilizing Peptide, DTT and ice cold MQ water) was added to the background wells. GS Red solution (7 μL; assay buffer 10×, Stabilizing Peptide, DTT, GS Red and ice cold water) was added to all wells except background wells. GR solution (7 μL; assay buffer 10×, Stabilizing Peptide, DTT, GR and ice cold water) was added to all wells. The plate was sealed and incubated in a dark at room temperature for 2 hours. The plate was read in an Analyst plate reader (LJL Biosystems/Molecular Devices Corporation) or other similar plate reader capable of recording fluorescence polarization (excitation wavelength 530 nm, emission wavelength 590 nm and a dichroic mirror at 561 nm). The IC₅₀ values were calculated using XLfit model 205 and are shown in Table 1.

TABLE 1

| | Inhibition of GR | | Inhibition of GR |
|---|---|---|---|
| Example No. | binding, IC₅₀ (nM) | Example No. | binding, IC₅₀ (nM) |
| 1 | 1.2 | 2 | 1.2 |
| 3 | 0.92 | 4 | 0.96 |
| 5 | 1.1 | 6 | 1.2 |
| 7 | 1 | 8 | 1.9 |
| 9 | 0.57 | 10 | 1.1 |
| 11 | 0.59 | 12 | 0.86 |
| 13 | 0.55 | 14 | 1.2 |
| 15 | 1 | 16 | 1.5 |
| 17 | 1.8 | 18 | 3.7 |
| 19 | 4.8 | 20 | 2.4 |
| 21 | 0.57 | 22 | 0.85 |
| 23 | 0.99 | 24 | 1.2 |
| 25 | 0.7 | 26 | 1.1 |
| 27 | 1.1 | 28 | 1.3 |
| 29 | 1.3 | 30 | 1.1 |
| 31 | 0.98 | 32 | 1 |
| 33 | 1 | 34 | 1 |
| 35 | 1.1 | 36 | 2.7 |
| 37 | 1.4 | 38 | 0.88 (8S)/2.2(8R) |
| 39 | 2.8 | 40 | 1.7 |
| 41 | 2.2 | 42 | 1.5 |
| 43 | 0.81 | 44 | 0.62 |

1. A compound of formula 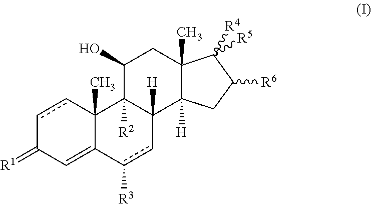
wherein
R¹ represents an oxygen atom;
R² represents a hydrogen, fluorine or chlorine atom;
R³ represents a hydrogen, fluorine or chlorine atom or a methyl group;
R⁴ represents —C(O)—Y—R⁷;
Y represents an oxygen or sulphur atom or a group >NR⁸;
R⁵ and R⁶ together with the carbon atoms to which they are attached form a 1,3-dioxolanyl group which is substituted by a 5- to 10-membered aromatic or heteroaromatic ring system optionally attached to the 1,3-dioxolanyl group via an alkylene, alkenylene or alkynylene linking group, the ring system itself being optionally substituted by one or more substituents independently selected from halogen, cyano, C₁-C₆ alkyl, C₁-C₆ alkoxy, trifluoromethyl and trifluoromethoxy;
R⁷ represents a C₁-C₆ alkyl, C₂-C₆ alkenyl or C₂-C₆ alkynyl group, each of which may be optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₘR⁹, —NHR¹⁰, and —NR¹¹R¹²;
m is 0, 1 or 2;
R⁸ represents a hydrogen atom, a group R⁷, or is linked to R⁷ to form a 3- to 8-membered, saturated or partially saturated heterocyclic ring optionally containing a further ring heteroatom selected from nitrogen, oxygen and sulphur, the heterocyclic ring being optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₙR¹³ and —NR¹⁴R¹⁵;
n is 0, 1 or 2;
R⁹, R¹⁰, R¹¹, R¹², R¹³, R¹⁴ and R¹⁵ each independently represent a C₁-C₆ alkyl group or an aryl group, each of which may be optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₚR¹⁶ and —NR¹⁷R¹⁸, and R¹⁴ and R¹⁵ may each additionally represent a hydrogen atom;
p is 0, 1 or 2; and
R¹⁶, R¹⁷ and R¹⁸ each independently represent a hydrogen atom or a C₁-C₆ alkyl group;
or a pharmaceutically acceptable salt thereof.

2. A compound according to claim 1, wherein
R¹ represents an oxygen atom;
R² represents a hydrogen, fluorine or chlorine atom;
R³ represents a hydrogen, fluorine or chlorine atom or a methyl group;
R⁴ represents —C(O)—Y—R⁷;
Y represents an oxygen or sulphur atom or a group >NR⁸;
R⁵ and R⁶ together with the carbon atoms to which they are attached form a 1,3-dioxolanyl group which is substituted by a 5- to 10-membered heteroaromatic ring system optionally attached to the 1,3-dioxolanyl group via an alkylene, alkenylene or alkynylene linking group;
R⁷ represents a C₁-C₆ alkyl, C₂-C₆ alkenyl or C₂-C₆ alkynyl group, each of which may be optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₘR⁹, —NHR¹⁰, and —NR¹¹R¹²;
m is 0, 1 or 2;
R⁸ represents a hydrogen atom, a group R⁷, or is linked to R⁷ to form a 3- to 8-membered, saturated or partially saturated heterocyclic ring optionally containing a further ring heteroatom selected from nitrogen, oxygen and sulphur, the heterocyclic ring being optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₙR¹³ and —NR¹⁴R¹⁵;
n is 0, 1 or 2;
R⁹, R¹⁰, R¹¹, R¹², R¹³, R¹⁴ and R¹⁵ each independently represent a C₁-C₆ alkyl group or an aryl group, each of which may be optionally substituted by one or more substituents independently selected from hydroxyl, halogen, cyano, nitro, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylcarbonyl, C₁-C₆ alkylcarbonyloxy, C₁-C₆ alkoxycarbonyl, —S(O)ₚR¹⁶ and —NR¹⁷R¹⁸, and R¹⁴ and R¹⁵ may each additionally represent a hydrogen atom;
p is 0, 1 or 2; and
R¹⁶, R¹⁷ and R¹⁸ each independently represent a hydrogen atom or a C₁-C₆ alkyl group;
or a pharmaceutically acceptable salt thereof.

3. A compound according to claim 1, wherein R² and R³ each represent a hydrogen atom or a fluorine atom.

4. A compound according to claim 1, wherein Y represents a sulphur atom.

5. A compound according to claim 1, wherein R⁷ represents a C₁-C₃ alkyl or C₂-C₄ alkynyl group, each of which may be optionally substituted by one or more substituents independently selected from hydroxyl, fluorine and cyano.

6. A compound according to claim 1, wherein R⁵ and R⁶ together with the carbon atoms to which they are attached form a 1,3-dioxolanyl group which is substituted by a 5- to 6-membered aromatic or heteroaromatic ring system, the ring system itself being optionally substituted by one or more substituents independently selected from halogen, cyano, C1-C6 alkyl, C1-C6 alkoxy, trifluoromethyl and trifluoromethoxy.

7. A compound according to claim 6, wherein the ring system is furanyl, thienyl, isoxazolyl or phenyl.

8. A compound according to claim 1 of formula (IB): 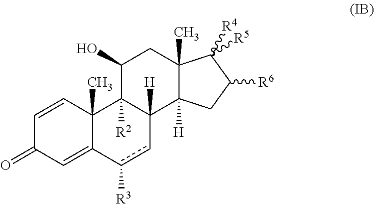
wherein R², R³, R⁴, R⁵ and R⁶ are as defined in claim 1.

9. A compound according to claim 1 being:
S-(Cyanomethyl)(4aS,4bR,6aS,6bS,9aR,10aS,10bS,12S)-4b,12-difluoro-8-(2-furyl)-4a,6a-dimethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carbothioate,
S-(4-Hydroxybut-2-yn-1-yl)(4aS,4bR,6aS,6bS,9aR,10aS,10bS,12S)-4b,12-difluoro-8-(2-furyl)-4a,6a-dimethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carbothioate,
S-(Fluoromethyl)(4aS,4bR,6aS,6bS,9aR,10aS,10bS,12S)-4b,12-difluoro-8-(2-furyl)-4a,6a-dimethyl-2-oxo-2,4a,4b,5,6,6a,9a,10,10a,10b,11,12-dodecahydro-6bH-naphtho[2′,1′:4,5]-indeno[1,2-d][1,3]dioxole-6b-carbothioate,
S-Methyl (4aS,4bR,5S,6aS,6bS,8S,9aR,10aS,10bS,12S)-4b,12-difluoro-5-hydroxy-4a,6a-dimethyl-2-oxo-8-(2-thienyl)-2,4a,4b,5,6,6a,9a,10,10a,10b,11,12-dodecahydro-6bH-naphtho[2′,1′:4,5]indeno[1,2-d][1,3]dioxole-6b-carbothioate,
S-Methyl (4aS,4bR,5S,6aS,6bS,9aR,10aS,10bS,12S)-8-(2,4-difluorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-8-(2,4-difluorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,8S,9aR,10aS,10bS,12S)-4b,12-difluoro-5-hydroxy-4a,6a-dimethyl-8-(5-methyl-2-furyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-Methyl (4aS,4bR,5S,6aS,6bS,9aR,10aS,10bS,12S)-4b,12-difluoro-5-hydroxy-4a,6a-dimethyl-8-(5-methyl-2-furyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,125)-4b,12-difluoro-5-hydroxy-4a,6a-dimethyl-2-oxo-8-(2-thienyl)-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-5-hydroxy-4a,6a-dimethyl-8-(5-methyl-2-furyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-8-(5-chloro-2-furyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-8-(1-benzofuran-2-yl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-8-(3-methyl-2-thienyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-2-oxo-8-[5-(trifluoromethyl)-2-furyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-2-oxo-8-(2-thienyl)-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-5-hydroxy-4a,6a-dimethyl-2-oxo-8-quinolin-6-yl-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-8-(4-methylphenyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-5-hydroxy-4a,6a-dimethyl-2-oxo-8-[4-(trifluoromethoxy)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-8-(2-methylphenyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-8-(3-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-5-hydroxy-4a,6a-dimethyl-8-(4-methylphenyl)-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-8-(3-cyanophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-8-(4-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-8-(4-chlorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-8-(4-chlorophenyl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-4b,12-difluoro-5-hydroxy-4a,6a-dimethyl-2-oxo-8-phenyl-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aS,4bR,5S,6aS,6bS,9aR,10aS,10bS,12S)-4b,12-difluoro-5-hydroxy-4a,6a-dimethyl-2-oxo-8-phenyl-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-8-(2,3-dihydro-1-benzofuran-7-yl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aS,4bR,5S,6aS,6bS,9aR,10aS,10bS,12S)-8-(3,5-dimethylisoxazol-4-yl)-4b,12-difluoro-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aR,4bS,5S,6aS,6bS,9aR,10aS,10bS)-8-furan-2-yl-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aR,4bS,5S,6aS,6bS,8R,9aR,10aS,10bS)-5-hydroxy-4a,6a-dimethyl-2-oxo-8-thiophen-2-yl-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aR,4bS,5S,6aS,6bS,9aR,10aS,10bS)-8-(2,4-dimethylphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aR,4bS,5S,6aS,6bS,9aR,10aS,10bS)-8-(2-fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-Methyl (4aR,4bS,5S,6aS,6bS,8R,9aR,10aS,10bS)-8-(2-fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-Methyl (4aR,4bS,5S,6aS,6bS,8S,9aR,10aS,10bS)-8-(2-fluoro-4-methoxyphenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aR,4bS,5S,6aS,6bS,9aR,10aS,10bS)-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aR,4bS,5S,6aS,6bS,9aR,10aS,10bS)-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-Methyl (4aR,4bS,5S,6aS,6bS,9aR,10aS,10bS)-5-hydroxy-4a,6a-dimethyl-2-oxo-8-[4-(trifluoromethyl)phenyl]-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Cyanomethyl)(4aR,4bS,5S,6aS,6bS,9aR,10aS,10bS)-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate,
S-(Fluoromethyl)(4aR,4bS,5S,6aS,6bS,9aR,10aS,10bS)-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate, or
S-Methyl (4aR,4bS,5S,6aS,6bS,9aR,10aS,10bS)-8-(2-fluorophenyl)-5-hydroxy-4a,6a-dimethyl-2-oxo-2,4a,4b,5,6,6a,9a,10,10a,10b,11,12-dodecahydro-6bH-naphtho[2′,1′:4,5]indeno[1,2-d][1,3]dioxole-6b-carbothioate.

10. A process for the preparation of a compound of formula (I) or a pharmaceutically acceptable salt thereof as defined in claim 1 which comprises
(i) reacting a compound of formula (II) 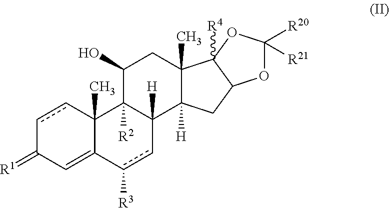
where R20 and R21 each independently represent a C₁-C₆ alkyl group and R¹, R², R³ and R⁴ are as defined in formula (I), with a compound of formula (III), OCH—X—R²², where X represents a bond or an alkylene, alkenylene or alkynylene linking group and R²² represents a 5- to 10-membered aromatic or heteroaromatic ring system optionally substituted by one or more substituents independently selected from halogen, cyano, C₁-C₆ alkyl, C₁-C₆ alkoxy, trifluoromethyl and trifluoromethoxy, or
(ii) when Y represents a sulphur atom, hydrolysing a compound of formula (IV) 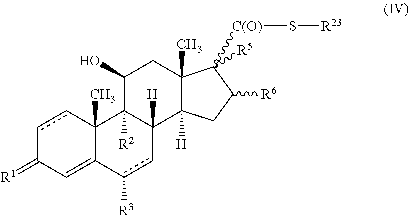
where R²³ represents a sulphur-protecting group and R¹, R², R³, R⁵ and R⁶ are as defined in formula (I), followed by reaction with a compound of formula (V), R⁷-L, where L represents a leaving group and R⁷ is as defined in formula (I), and optionally thereafter carrying out one or more of the following procedures:
converting a compound of formula (I) into another compound of formula (I)
removing any protecting groups
forming a pharmaceutically acceptable salt.

11. A pharmaceutical composition comprising a compound of formula (I) as claimed in claim 1 or a pharmaceutically acceptable salt thereof in association with a pharmaceutically acceptable adjuvant, diluent or carrier.

12. A compound of formula (I) as claimed in claim 1 or a pharmaceutically acceptable salt thereof for use in treating asthma, chronic obstructive pulmonary disease or allergic rhinitis.

13. A method of treating an obstructive airways disease in a patient suffering from, or at risk of, said disease, which comprises administering to the patient a therapeutically effective amount of a compound of formula (I) as claimed in claim 1 or a pharmaceutically acceptable salt thereof.

14. A combination of a compound of formula (I) as claimed in claim 1 or a pharmaceutically acceptable salt thereof and one or more agents independently selected from:
a selective β₂ adrenoceptor agonist;
a phosphodiesterase inhibitor;
a protease inhibitor;
an anticholinergic agent;
a modulator of chemokine receptor function; and
an inhibitor of kinase function.
